(19) United States
(12) Patent Application Publication
(10) Pub. No.: US 2026/0224528 A1
(43) Pub. Date: Aug. 6, 2026

(54) PSILOCIN FORMULATIONS

(71) Applicant: Eleusis Therapeutics US, Inc., NEW YORK, NY (US)

(72) Inventors: Robert Russell CONLEY, Annapolis, MD (US); Yonatan WEISS, Raanana (IL); Esmira NAFTALI, Petach Tikva (IL)

(21) Appl. No.: 19/154,039

(22) PCT Filed: Feb. 8, 2024
(86) PCT No.: PCT/IB2024/051166

§ 371 Date: Aug. 6, 2025

Related U.S. Application Data
(60) Provisional application No. 63/444,027, filed on Feb. 8, 2023.

(30) Foreign Application Priority Data
Feb. 24, 2023 (GB) 2302750.1

(51) Int. Cl. A61K 31/4045; A61K 9/00; A61K 9/08; A61K 9/16; A61K 9/19

(52) U.S. Cl. CPC A61K 31/4045; A61K 9/0019; A61K 9/08; A61K 9/1611; A61K 9/1617; A61K 9/1623; A61K 9/1652; A61K 9/19

(57) ABSTRACT

The invention features lyophilized solid formulations of psilocin and uses thereof. The disclosure provides a lyophilized solid comprising (i) amorphous psilocin, or a pharmaceutically acceptable salt thereof; (ii) citrate buffer as a buffering agent; (iii) mannitol as a bulking agent; and (iv) less than 2% (w/w) water.

### BACKGROUND

Significant interest in the therapeutic application of psilocin has developed, based upon evidence of possible therapeutic effects in a wide array of clinical applications, including psychiatric conditions, pain disorders, and neurological conditions. However, due to the physical properties of psilocin in the solid state, e.g., poor crystallinity with limited enhancement of bulk purity upon crystallization, susceptibility to auto catalyzed oxidation upon handling and prolonged storage, and low water solubility. Therefore, there exists a need for psilocin salts and formulations with improved stability, physical properties, and handling characteristics.

### SUMMARY OF THE INVENTION

In an aspect, the disclosure provides a lyophilized solid comprising (i) amorphous psilocin, or a pharmaceutically acceptable salt thereof; (ii) citrate buffer as a buffering agent; (iii) mannitol as a bulking agent; and (iv) less than 2% (w/w) water.

In some embodiments, the pharmaceutically acceptable salt thereof of psilocin may be the benzoate, succinate, tartrate, 1,5-naphthalenedisulfonate, stearate, lactate, acetate, aspartate, benzenesulfonate, besylate, bicarbonate, bitartrate, bromide, camsylate, carbonate, chloride, citrate, decanoate, edetate, estolate, esylate, fumarate, gluceptate, gluconate, glutamate, glycolate, glycollylarsanilate, hexanoate, hexylresorcinate, hydrabamine, hydroxynaphthoate, iodide, isethionate, lactate, lactobionate, malate, maleate, mandelate, mesylate, methylbromide, methylnitrate, methylsulfate, mucate, napsylate, nitrate, octanoate, oleate, pamoate, pantothenate, phosphate, polygalacturonate, propionate, salicylate, stearate, subacetate, sulfate, teoclate, tosylate, and triethiodide.

In an aspect, the disclosure provides a lyophilized solid comprising (i) amorphous psilocin benzoate; (ii) citrate buffer as a buffering agent; (iii) mannitol as a bulking agent; and (iv) less than 2% (w/w) water.

In an aspect, the disclosure provides a lyophilized solid comprising (i) an amorphous acid addition salt of psilocin benzoate; (ii) a buffering agent; and (iii) less than 2% (w/w) water.

For example, the freebase of psilocin when reacted with benzoic acid would give an acid addition salt of psilocin benzoate. In an embodiment, there is provided a lyophilized solid comprising an amorphous acid addition salt of psilocin, wherein the acid addition salt is psilocin benzoate. In an embodiment, there is provided a lyophilized solid comprising (i) an amorphous acid addition salt of psilocin, wherein the acid addition salt is psilocin benzoate; (ii) a buffering agent; and (iii) less than 2% (w/w) water.

In another aspect, the disclosure provides a lyophilized solid comprising (i) an amorphous acid addition salt of psilocin benzoate; (ii) a buffering agent; and (iii) less than 3% (w/w) water. In some embodiments, the psilocin benzoate has a concentration of between 0.01% (w/w) and 10% (w/w) (e.g., between 0.01% (w/w) and 9% (w/w), 0.01% (w/w) and 8% (w/w), 0.01% (w/w) and 7% (w/w), 0.01% (w/w) and 6% (w/w), 0.01% (w/w) and 5% (w/w), 0.01% (w/w) and 4% (w/w), 0.01% (w/w) and 3% (w/w), 0.01% (w/w), 0.01% (w/w) and 1% (w/w), 0.01% (w/w) and 0.5% (w/w), 0.01% (w/w) and 0.05% (w/w), 0.05% and 10% (w/w), 0.5% (w/w) and 10% (w/w), 1% (w/w) and 10% (w/w), 2% (w/w) and 10% (w/w), 3% (w/w) and 10% (w/w), 4% (w/w) and 10% (w/w), 5% (w/w) and 10% (w/w), 6% (w/w) and 10% (w/w), 7% (w/w) and 10% (w/w), 8% (w/w) and 10% (w/w), or 9% (w/w) and 10% (w/w)).

The solid may comprises less than 80% (w/w) (e.g., less than 70% (w/w), less than 60% (w/w), less than 50% (w/w), less than 40% (w/w), less than 30% (w/w), less than 20% (w/w), or less than 10% (w/w)) of a buffering agent. In some embodiments, the solid comprises between 10% (w/w) and 95% (w/w) (e.g., between 10% (w/w) and 90% (w/w), 10% (w/w) and 80% (w/w), 10% (w/w) and 70% (w/w), 10% (w/w) and 60% (w/w), 10% (w/w) and 50% (w/w), 10% (w/w) and 40% (w/w), 10% (w/w) and 30% (w/w), 10% (w/w) and 20% (w/w), 20% (w/w) and 95% (w/w), 30% (w/w) and 95% (w/w), 40% (w/w) and 95% (w/w), 50% (w/w) and 95% (w/w), 60% (w/w) and 95% (w/w), 70% (w/w) and 95% (w/w), or 80% (w/w) and 95% (w/w)) buffering agent. For example, the solid may include between 10% (w/w) and 30% (w/w) (e.g., 10% (w/w)±2% (w/w), 12% (w/w)±2% (w/w), 14% (w/w)±2% (w/w), 16% (w/w)±2% (w/w), 18% (w/w)±2% (w/w), 20% (w/w)±2% (w/w), 22% (w/w)±2% (w/w), 24% (w/w)±2% (w/w), 26% (w/w)±2% (w/w), 28% (w/w)±2% (w/w), or 30% (w/w)±2% (w/w)) buffering agent.

The buffering agent may be a citrate buffer.

In some embodiments, the solid further comprises a bulking agent. The bulking agent may have a concentration of between 65% (w/w) and 95% (w/w) (e.g., between 65% (w/w) and 90% (w/w), 65% (w/w) and 85% (w/w), 65% (w/w) and 80% (w/w), 65% (w/w) and 75% (w/w), 65% (w/w) and 70% (w/w), 70% (w/w) and 95% (w/w), 75% (w/w) and 95% (w/w), 80% (w/w) and 95% (w/w), 85% (w/w) and 95% (w/w), or 90% (w/w) and 95% (w/w)). For example, the bulking agent has a concentration of between 70% (w/w) and 90% (w/w) (e.g., 70% (w/w)±2% (w/w), 72% (w/w)±2% (w/w), 74% (w/w)±2% (w/w), 76% (w/w)±2% (w/w), 78% (w/w)±2% (w/w), 80% (w/w)±2% (w/w), 82% (w/w)±2% (w/w), 84% (w/w)±2% (w/w), 86% (w/w)±2% (w/w), 88% (w/w)±2% (w/w) or 90% (w/w)±2% (w/w)). In certain embodiments, the bulking agent is mannitol.

In another aspect, the disclosure provides a lyophilized solid comprising (i) an amorphous psilocin benzoate in an amount of between 0.01% (w/w) and 10% (w/w) (e.g., between 0.01% (w/w) and 8% (w/w), 0.01% (w/w) and 6% (w/w), 0.01% (w/w) and 4% (w/w), 0.01% (w/w) and 2% (w/w), 0.01% (w/w) and 1% (w/w), 1% (w/w) and 10% (w/w), 2% (w/w) and 10% (w/w), 3% (w/w) and 10% (w/w), 4% (w/w) and 10% (w/w), 5% (w/w) and 10% (w/w), 6% (w/w) and 10% (w/w), 7% (w/w) and 10% (w/w), 8% (w/w) and 10% (w/w), or 9% (w/w) and 10% (w/w)), (ii) a buffering agent in an amount of between 10% (w/w) and 30% (w/w) (e.g., 10% (w/w)±2% (w/w), 12% (w/w)±2% (w/w), 14% (w/w)±2% (w/w), 16% (w/w)±2% (w/w), 18% (w/w)±2% (w/w), 20% (w/w)±2% (w/w), 22% (w/w)±2% (w/w), 24% (w/w)±2% (w/w), 26% (w/w)±2% (w/w), 28% (w/w)±2% (w/w), or 30% (w/w)±2% (w/w)); (iii) a bulking agent in an amount of between 70% (w/w) and 95% (w/w) (e.g., 70% (w/w)±2% (w/w), 72% (w/w)±2% (w/w), 74% (w/w)±2% (w/w), 76% (w/w)±2% (w/w), 78% (w/w)±2% (w/w), 80% (w/w)±2% (w/w), 82% (w/w)±2% (w/w), 84% (w/w)±2% (w/w), 86% (w/w)±2% (w/w), 88% (w/w)±2% (w/w), 90% (w/w)±2% (w/w), 92% (w/w)±2% (w/w), 94% (w/w)±2% (w/w), or 95% (w/w)±2% (w/w)); and (iii) less than 3% (w/w) water (e.g., less than 2.5% (w/w), 2% (w/w), 1.5% (w/w), 1% (w/w), 0.5% (w/w), 0.05% (w/w)).

In some embodiments, the buffer is a citrate buffer. In certain embodiments, the bulking agent comprises mannitol. In some embodiments, the solid comprises less than 1% (w/w) (e.g., less than 0.9% (w/w), less than 0.8% (w/w), less than 0.7% (w/w), less than 0.6% (w/w), less than 0.5% (w/w), less than 0.4% (w/w), less than 0.3% (w/w), less than 0.2% (w/w), less than 0.1% (w/w), less than 0.05% (w/w)) water.

The lyophilized solid may have a pH of between 3.5 and 5 (e.g., pH of between 3.5 and 5, 4 and 5, 4.5 and 5, 3.5 and 4.5, or 4 and 4.5) when dissolved in between 10 mL and 50 mL (e.g., between 10 mL and 40 mL, 10 mL and 30 mL, 10 mL and 20 mL, 20 mL and 50 mL, 30 mL and 50 mL, or 40 mL and 50 mL) of unbuffered water or saline for injection.

In some embodiments, the solid further comprises less than 1% (w/w) of an antioxidant. The antioxidant may be sodium bisulfite. In certain embodiments, the solid is free of any antioxidant.

The solid may comprise less than 20% (w/w) by weight of sodium chloride. In some embodiments, the solid is free of sodium chloride. In some embodiments, the lyophilized solid does not comprise mannitol hemihydrate. In some embodiments, the lyophilized solid is substantially free of mannitol hemihydrate. In some embodiments, the lyophilized solid is substantially free of mannitol hemihydrate and wherein the lyophilized solid is characterized by an X-ray powder diffractogram (XRPD) which does not comprise a peak at the 2θ values of 17.9±0.1°, 17.9±0.2° or 17.9±0.3° as measured using an X-ray wavelength of 1.5406 A.

In another aspect, the disclosure provides a method of preparing a lyophilized solid comprising (i) an amorphous acid addition salt of psilocin benzoate; (ii) a buffering agent; and (iii) less than 3% (w/w) water, wherein the solid described herein is dissolved in between 5 mL to 50 mL (e.g., between 5 mL and 40 mL, 5 mL and 30 mL, 5 mL and 20 mL, 5 mL and 10 mL, 10 mL and 50 mL, 20 mL and 50 mL, 30 mL and 50 mL, or 40 mL and 50 mL) of an aqueous solution and undergoes a thermal treatment step, followed by a drying step. In some embodiments, the thermal treatment step is performed for between 30 hours and 70 hours (e.g., between 30 hours and 60 hours, 30 hours and 50 hours, 30 hours and 40 hours, 40 hours and 70 hours, 50 hours and 70 hours, or 60 hours and 70 hours) at a temperature of between −50° C. and 10° C. (e.g., between −50° C. and 0° C., −50° C. and −10° C., −50° C. and −20° C., −50° C. and −30° C., −50° C. and −40° C., −40° C. and 10° C., −30° C. and 10° C., −20° C. and 10° C., −10° C. and 10° C., or 0° C. and 10° C.). In some embodiments, the thermal treatment step comprises between 3 and 8 steps (e.g., 3, 4, 5, 6, 7, or 8 steps).

The thermal treatment step may include (i) a first step, wherein the first step is performed at about 5° C. for about 10 minutes; (ii) a second step, wherein the second step is performed at about −40° C. for about 45 minutes; (iii) a third step, wherein the third step is performed at about −40° C. for about 120 minutes; (iv) a fourth step, wherein the fourth step is performed at about −9° C. for about 31 minutes; (v) a fifth step, wherein the fifth step is performed at about −9° C. for about 300 minutes; (vi) a sixth step, wherein the sixth step is performed at about −40° C. for about 31 minutes; and (vii) a seventh step, wherein the seventh step is performed at about −40° C. for about 120 minutes.

In certain embodiments, the drying step comprises a primary drying step and a secondary drying step. In some embodiments, the primary drying step is performed for between 5 hours and 10 hours (e.g., between 5 hours and 8 hours, 5 hours and 6 hours, 6 hours and 10 hours, or 8 hours and 10 hours) at a temperature of between −50° C. and 0° C. (e.g., between −50° C. and −10° C., −50° C. and −20° C., −50° C. and −30° C., −50° C. and −40° C., −40° C. and 0° C., −30° C. and 0° C., −20° C. and 0° C., or −10° C. and 0° C.), and a pressure of between 20 μBar and 300 μBar (e.g., between 20 μBar and 100 μBar, 20 μBar and 200 μBar, 20 μBar and 300 μBar, 100 μBar and 300 μBar, 100 μBar and 200 μBar, or 50 μBar and 200 μBar).

The primary drying step may comprises a first step, a second step, and a third step, wherein the first step is performed at about −40° C. for about 60 minutes with a pressure of about 50 μBar, wherein the second step is performed at about −9° C. for about 31 minutes with a pressure of about 200 μBar, and wherein the third step is performed at −9° C. for about 2566 minutes with a pressure of about 200 μBar. The secondary dry step may comprises a first step and second step, wherein the first step is performed at 25° C. for about 78 minutes at a pressure of about 50 μB Tar and wherein the second step is performed at 25° C. for about 480 minutes at a pressure of about 50 μBar.

In another aspect, the disclosure provides a method of treating a disease or condition in a subject in need thereof including intravenously administering to the subject any one of the lyophilized solids described herein dissolved in from 5 mL to 50 mL (e.g., between 5 mL and 40 mL, 5 mL and 30 mL, 5 mL and 20 mL, 5 mL and 10 mL, 10 mL and 50 mL, 20 mL and 50 mL, 30 mL and 50 mL, or 40 mL and 50 mL) of an aqueous solution in an amount sufficient to treat the disease or condition.

### BRIEF DESCRIPTION OF THE DRAWINGS

FIG. 1 shows XRD patterns of lyophiles obtained from a formulation containing 6 mg psilocin benzoate, 60 mg mannitol and in 25 mM citrate buffer (pH=4.5). The observation of a peak at 17.9 is characteristic of mannitol hemihydrate.

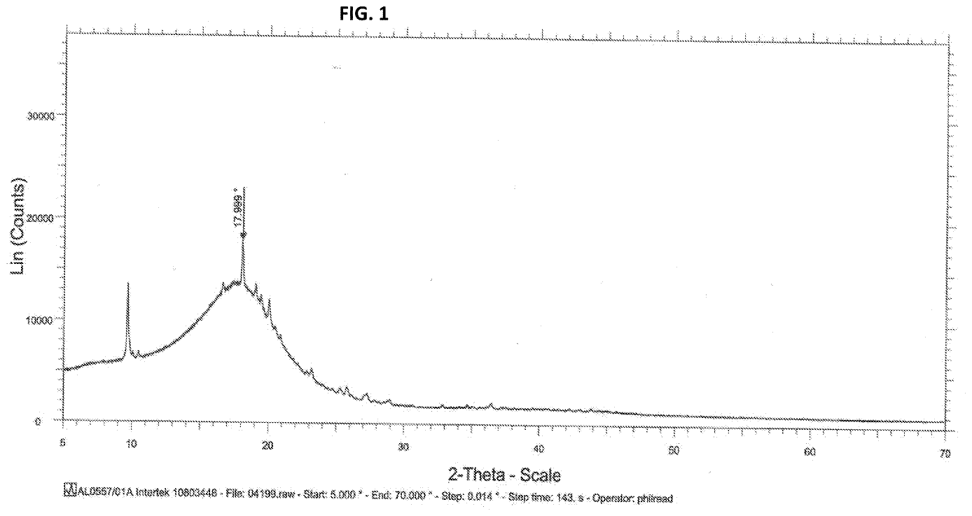

FIG. 2 shows modulated differential scanning calorimetry (MDSC) analysis thermograms obtained from formulations CF1A1 (mannitol based) from optimization cycle 2.

FIG. 3 shows MDSC analysis thermograms obtained from formulations of CF1A2 (mannitol and lactose based) from optimization cycle 2.

FIG. 4 shows a representative freeze-drying chart from optimization cycle 2.

### DEFINITIONS

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

Terms such as “a”, “an,” and “the” are not intended to refer to only a singular entity but include the general class of which a specific example may be used for illustration.

As used herein, the term “about” refers to a value that is within 10% above or below the value being described.

As used herein, the terms “acute stress disorder” and “ASD” refer to a condition that arises as a response to a stressful event or situation of an exceptionally threatening or catastrophic nature, which is likely to cause pervasive distress in an individual (e.g., natural or man-made disaster, combat, serious accident, witnessing the violent death of others, or being the victim of torture, terrorism, rape, or other crime). Like PTSD, acute stress disorder is an anxiety disorder that involves a very specific reaction following exposure to a traumatic event or stressor. However, the duration of acute stress disorder is shorter than that for PTSD, such that the symptoms are present for at least one, two, or three days, but no more than four, five, or six weeks. For individuals exhibiting symptoms persisting for a longer period of time, a diagnosis of PTSD may be warranted.

The term “administration” or “administering” refers to a method of giving a dosage of a compound or pharmaceutical composition to a subject.

By “dysthymia” or “dysthymic disorder” is meant a chronically depressed mood that occurs for most of the day, more days than not, for at least two years. In children and adolescents, the mood may be irritable rather than depressed, and the required minimum duration is one year. During the two-year period (one year for children or adolescents), any symptom-free intervals last no longer than 2 months. During periods of depressed mood, at least two of the following additional symptoms are present: poor appetite or overeating, insomnia or hypersomnia, low energy or fatigue, low self-esteem, poor concentration, or difficulty making decisions, and feelings of hopelessness. The symptoms cause clinically significant distress or impairment in social, occupational (or academic), or other important areas of functioning. The diagnosis of dysthymia is not made if: the individual has ever had a manic episode, a mixed episode, a hypomanic episode; has ever met the criteria for a cyclothymic disorder; the depressive symptoms occur exclusively during the course of a chronic psychotic disorder (e.g., schizophrenia); or if the disturbance is due to the direct physiological effects of a substance or a general medical condition. After the initial two-years of dysthymic disorder, major depressive episodes may be superimposed on the dysthymic disorder (“double depression”). Diagnostic and Statistical Manual of Mental Disorders (OSM IV), American Psychiatric Press, 4th Edition, I 994. Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, VA.; American Psychiatric Association, 2013).

By “free base equivalent” is meant an amount corresponding to a free base equivalent in a mass of psilocin benzoate. For example, a free base equivalent of 1 mg of psilocin is equal to 1 mg of psilocin in its free base form and equal to 1.60 mg of psilocin in its benzoate salt form (e.g., 1.0×(326.39/204.27) to account for the mass contribution of the benzoic acid).

As used herein, the term “generalized anxiety disorder” refers to a condition characterized by excessive anxiety and worry (i.e., apprehensive expectation). Typically, the excessive anxiety and worry occur on more days than not for a period of time (e.g., one, two, three, or four months or more). The anxiety and worry can be associated with (i) restlessness, feeling keyed up, or on edge; and/or (ii) muscle tension. The anxiety and worry can be associated with (a) a marked avoidance of situations in which a negative outcome could occur; (b) a marked time and effort preparing for situations in which a negative outcome could occur; (c) a marked procrastination in behavior or decision-making due to worries; and (d) repeatedly seeking reassurance due to worries. The anxiety, worry, or physical symptoms can cause clinically significant distress or impairment in social, occupational, or other important areas of functioning in many, but not necessarily all individuals with GAD.

As used herein, the terms “obsessive compulsive disorder,” “OCD,” and “anxiety and obsessive-compulsive spectrum disorders” refer to a condition characterized by obsessions and/or compulsions. Obsessions are recurrent and persistent thoughts, urges, or images that are experienced, at some time during the disturbance, as intrusive and unwanted and that usually cause marked anxiety or distress in which the obsessed individual attempts to ignore or suppress such thoughts, urges, or images, or to neutralize them with some other thought or action (i.e., by performing a compulsion). Compulsions are repetitive behaviors (e.g., hand washing, ordering, checking) or mental acts (e.g., praying, counting, repeating words silently) that the person feels driven to perform in response to an obsession, or according to rules that must be applied rigidly. The behaviors or mental acts are aimed at preventing or reducing anxiety or distress, or preventing some dreaded event or situation; however, these behaviors or mental acts either are not connected in a realistic way with what they are designed to neutralize or prevent, or are clearly excessive. Typically the obsessions or compulsions are time consuming (for example, take more than 1 hour a day), or cause clinically significant distress or impairment in social, occupational, or other important areas of functioning.

As used herein, the term “panic disorder” refers to a condition characterized by recurrent and unexpected panic attacks. Panic disorder includes both panic disorder with agoraphobia and panic disorder without agoraphobia. Subjects with this condition can exhibit one or both of the following: (i) a persistent concern or worry about additional panic attacks or their consequences (e.g., losing control, having a heart attack, going crazy); and/or (ii) significant maladaptive change in behavior related to the attacks (e.g., behaviors designed to avoid having panic attacks), which may include agoraphobic avoidance.

As used herein, the terms “pharmacologically effective amount,” “therapeutically effective amount,” and the like, when used in reference to a therapeutic composition, refer to a quantity sufficient to, when administered to the subject, including a mammal, for example a human, effect beneficial or desired results, such as clinical results. For example, in the context of treating depression, described herein, these terms refer to an amount of the composition sufficient to achieve a treatment response as compared to the response obtained without administration of the composition. The quantity of a given composition described herein that will correspond to such an amount may vary depending upon various factors, such as the given agent, the pharmaceutical formulation, the route of administration, the type of disease or disorder, the identity of the subject (e.g., age, sex, weight) or host being treated, and the like. An “effective amount,” “pharmacologically effective amount,” or the like, of a composition of the present disclosure, also include an amount that results in a beneficial or desired result in a subject as compared to a control (e.g., a decrease in the score on the Montgomery-Asberg Depression Rating Scale).

As used herein, the terms “post traumatic stress disorder” and “PTSD” refer to a condition that arises as a delayed and/or protracted response to a stressful event or situation (either short- or long-lasting) of an exceptionally threatening or catastrophic nature, which is likely to cause pervasive distress in an individual (e.g., natural or man-made disaster, combat, serious accident, witnessing the violent death of others, or being the victim of torture, terrorism, rape, or other crime). Predisposing factors such as personality traits (e.g., compulsive, asthenic) or previous history of neurotic illness may lower the threshold for the development of the condition or aggravate its course, but they are neither necessary nor sufficient to explain its occurrence. PTSD is a less frequent and more enduring consequence of psychological trauma than the more frequently seen acute stress response. PTSD has been recognized in the past as railway spine, stress syndrome, shell shock, battle fatigue, traumatic war neurosis, and post-traumatic stress syndrome. Diagnostic symptoms include re-experiencing original trauma(s), by means of flashbacks or nightmares; avoidance of stimuli associated with the trauma; and increased arousal, such as difficulty falling or staying asleep, anger, and hypervigilance. Formal diagnostic criteria (DSM-V, DSM-IV, and/or ICD-9) require that the symptoms last more than one month and cause significant impairment in social, occupational, or other important areas of functioning (e.g., problems with work and/or relationships). Formal diagnostic criteria can include: (i) intrusion symptoms that are associated with the traumatic event (e.g., (a) spontaneous or cued recurrent, involuntary, and intrusive distressing memories of the traumatic event; (b) recurrent distressing dreams in which the content and/or affect of the dream is related to the event; (c) dissociative reactions (e.g., flashbacks) in which the individual feels or acts as if the traumatic event were recurring (such reactions may occur on a continuum, with the most extreme expression being a complete loss of awareness of present surroundings; (d) intense or prolonged psychological distress at exposure to internal or external cues that symbolize or resemble an aspect of the traumatic event; and/or (e) marked physiological reactions to reminders of the traumatic event); (ii) persistent avoidance of stimuli associated with the traumatic event (e.g., (a) thoughts, feelings, or physical sensations that arouse recollections of the traumatic event; (b) activities, places, physical reminders, or times (e.g., anniversary reactions) that arouse recollections of the traumatic event; and/or (c) people, conversations, or interpersonal situations that arouse recollections of the traumatic event); (iii) negative alterations in cognitions and mood that are associated with the traumatic event (e.g., (a) inability to remember an important aspect of the traumatic event (typically dissociative amnesia); (b) persistent and exaggerated negative expectations about one's self, others, or the world; (c) persistent distorted blame of self or others about the cause or consequences of the traumatic event; (d) pervasive negative emotional state (e.g., fear, horror, anger, guilt, or shame); (e) markedly diminished interest or participation in significant activities; (f) feeling of detachment or estrangement from others; and/or (g) persistent inability to experience positive emotions (e.g., unable to have loving feelings, psychic numbing); and (iv) alterations in arousal (i.e., hyperarousal) and reactivity that are associated with the traumatic event (e.g., (a) irritable, angry, or aggressive behavior; (b) reckless or self-destructive behavior; (c) hypervigilance; (d) exaggerated startle response; (e) problems with concentration; and/or (f) sleep disturbance (e.g., difficulty falling or staying asleep, or restless sleep)). Formal diagnostic criteria can further include that the duration of disturbance is more than a certain period of time (e.g., one month, three months, or six months) and that the disturbance causes clinically significant distress or impairment in social, occupational, or other important areas of functioning. In a small proportion of patients the condition may show a chronic course over many years and a transition to an enduring personality change. The three main symptoms associated with PTSD are (1) “reliving” the traumatic event, such as flashbacks, nightmares, intrusive thoughts and recollections, (2) avoidance behaviors and emotional numbing, and (3) hypersensitivity such as an inability to sleep, anxious feelings, overactive startle response, hyperarousal, hypervigilance, irritability, and outbursts of anger.

As used herein, the terms “psychological disorder” and “psychological condition” refer to a condition characterized by a disturbance in one's emotional or behavioral regulation that reflects a dysfunction in the psychological, biological, or developmental processes underlying mental function. Psychological disorders include, but are not limited to depressive disorders (major depression, treatment resistant depression, melancholic depression, atypical depression, or dysthymia), anxiety disorders (end of life anxiety, generalized anxiety disorder, panic disorder, social anxiety, post-traumatic stress disorder, acute stress disorder, obsessive compulsive disorder, or social phobia), addictions (e.g., substance abuse, e.g., alcoholism, tobacco abuse, or drug abuse)), eating disorders (e.g., anorexia nervosa, bulimia nervosa, and binge eating disorder) and compulsive behavior disorders (e.g., primary impulse-control disorders or obsessive-compulsive disorder). Psychological disorders can be any psychological condition associated with one or more symptoms, e.g., somatic symptoms (e.g., chronic pain, anxiety disproportionate to severity of physical complaints, pain disorder, body dysmorphia, conversion (i.e., loss of bodily function due to anxiety), hysteria, or neurological conditions without identifiable cause), or psychosomatic symptoms (e.g., back pain, fibromyalgia, migraines, and chronic fatigue syndrome). Psychological disorders also include repetitive body-focused behaviors, such as tic disorders (e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder). In some cases, development of a psychological disorder is associated with or characterized by a prodromal symptom, such as depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

As used herein, the terms “social phobia” and “social anxiety disorder” refer to a condition characterized by fear or anxiety associated with one or more social situations. Subjects with this condition typically exhibit a marked fear or anxiety about one or more social situations in which the person is exposed to possible scrutiny by others. Examples include social interactions (e.g., having a conversation), being observed (e.g., eating or drinking), or performance in front of others (e.g., giving a speech). Typically, an individual with this condition (i) fears that he or she will act in a way, or show anxiety symptoms that will be negatively evaluated (i.e., be humiliating, embarrassing, lead to rejection, or offend others); (ii) the social situations almost invariably provoke immediate fear or anxiety; (iii) the social situations are avoided or endured with intense fear or anxiety; and (iv) the fear or anxiety is out of proportion to the danger posed by the social situation. In children, the fear or anxiety may be expressed by crying, tantrums, freezing, clinging, shrinking or refusal to speak in social situations. The fear, anxiety, and avoidance can cause clinically significant distress or impairment in social, occupational, or other important areas of functioning.

As used herein, the terms “treat,” “treating,” or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (e.g., by reducing one or more symptoms of inflammation). The term “therapeutic” includes the effect of mitigating deleterious clinical effects of certain inflammatory processes (i.e., consequences of the inflammation, rather than the symptoms of inflammation). The methods of the invention can be used as a primary prevention measure, i.e., to prevent a condition or to reduce the risk of developing a condition. Prevention refers to prophylactic treatment of a patient who may not have fully developed a condition or disorder, but who is susceptible to, or otherwise at risk of, the condition. Thus, in the claims and embodiments, the methods of the invention can be used either for therapeutic or prophylactic purposes.

By “major depressive disorder” is meant a clinical course that is characterized by one or more major depressive episodes in an individual without a history of manic, mixed, or hypomanic episodes. The diagnosis of unipolar depression is not made if: manic, mixed, or hypomanic episodes develop during the course of depression; if the depression is due to the direct physiological effects of a substance; if the depression is due to the direct physiological effects of a general medical condition; if the depression is due to a bereavement or other significant loss (“reactive depression”); or if the episodes are better accounted for by schizoaffective disorder and are not superimposed on schizophrenia, schizophreniform disorder, delusional disorder, or psychotic disorder. If manic, mixed, or hypomanic episodes develop, then the diagnosis is changed to a bipolar disorder. Depression may be associated with chronic general medical conditions (e.g., diabetes, myocardial infarction, carcinoma, and stroke). Generally, unipolar depression is more severe than dysthymia. The essential feature of a major depressive episode is a period of at least two to 15 weeks during which there is either depressed mood or loss of interest or pleasure in nearly all activities. In children and adolescents, the mood may be irritable rather than sad. The episode may be a single episode or may be recurrent. The individual also experiences at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation, plans, or attempts. Each symptom must be newly present or must have clearly worsened compared with the person's pre-episode status. The symptoms must persist for most of the day, nearly every day, for at least two consecutive weeks, and the episode must be accompanied by clinically significant distress or impairment in social, occupational (or academic), or other important areas of functioning (Diagnostic and Statistical Manual of Mental Disorders (OSM IV), American Psychiatric Press, 4th Edition, 1994). Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, VA.; American Psychiatric Association, 2013).

Other features and advantages of the invention will be apparent from the following Detailed Description, Examples, and Claims.

### DETAILED DESCRIPTION OF THE INVENTION

The disclosure provides psilocin formulations including psilocin benzoate salt having less than 2% (w/w) water. The invention provides for improved formulations and uses of psilocin benzoate salt. For example, the psilocin formulations described herein may include psilocin benzoate, a buffering agent, less than 2% (w/w) water, and optionally a bulking agent. The psilocin benzoate formulations described herein are lyophilized solids which are suitable to be dissolved rapidly in a diluent for intravenous infusion. Furthermore, described herein are methods of producing the lyophilized solids described herein using a drying protocol. The psilocin benzoate salt formulations can exhibit improved shelf-life stability.

The invention further features methods of treating psychological conditions, neurological injuries, pain, cephalic pain (e.g., headache), inflammatory conditions, and anxiety in a subject by utilizing the intravenous psilocin benzoate infusion formulations. The psilocin benzoate formulations may be administered in combination with another therapeutic agent, such as an antiemetic, a benzodiazepine, and/or an anti-inflammatory agent. The psilocin benzoate formulations may be administered in combination with a virtual reality (VR), augmented reality (AR), spatial computing (SC) and/or electronic device experience. Said experience may take place prior to, during and/or after the administration of the psilocin benzoate formulation.

### Psilocin

Psilocin has the structure:

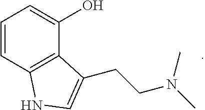

Psilocybin is a phosphate prodrug for psilocin. Psilocin is much more lipid soluble in comparison to psilocybin, and therefore is capable of crossing the blood brain barrier more effectively to elicit a response. Psilocin has a high affinity for and is able to activate the 5-HT2A receptor, which plays a key role in regulating mood, sexual behavior, aggression, impulsivity, cognitive function, appetite, pain, sleep, and memory along with other behaviors. As result, psilocin has effects at the 5-HT2A receptor that mimic the action of the endogenous neurotransmitter serotonin. This disclosure provides methods for intravenous administration of psilocin benzoate that are useful in therapy, such as in the treatment of a patient having a psychological condition or a neurological injury. The psilocin benzoate described herein may be a 1:1 benzoate salt.

### Compositions

The invention features lyophilized solids of psilocin including a buffering agent and including less than 2% (w/w) water. The lyophilized solids described herein may include one or more pharmaceutically acceptable excipients. Examples of pharmaceutically acceptable excipients include, but are not limited to, biocompatible vehicles, adjuvants, additives, and diluents to achieve a composition usable as a dosage form.

The lyophilized solids described herein may include less than 3% (w/w) water (e.g., less than 2.5% (w/w), less than 2% (w/w), less than 1% (w/w), less than 0.5% (w/w), or less than 0.1% (w/w)). For example, the solids of the invention may include less than 1% (w/w) (e.g., less than 0.9% (w/w), less than 0.8% (w/w), less than 0.7% (w/w), less than 0.6% (w/w), less than 0.5% (w/w), less than 0.4% (w/w), less than 0.3% (w/w), less than 0.2% (w/w), or less than 0.1% (w/w)) water. In some embodiments, the lyophilized solid includes between 0.1% (w/w) and 1% (w/w) (e.g., between 0.1% (w/w) and 0.8% (w/w), 0.1% (w/w) and 0.6% (w/w), 0.1% (w/w) and 0.4% (w/w), 0.1% (w/w) and 0.2% (w/w), 0.2% (w/w) and 1% (w/w), 0.4% (w/w) and 1% (w/w), 0.6% (w/w) and 1% (w/w), or 0.8% (w/w) and 1% (w/w)).

The pharmaceutical compositions of the invention may be in the form of a powder that can be reconstituted (e.g., a lyophilized powder). In some embodiments, the composition can be stored as a lyophilized powder that is reconstituted prior to administration to a subject. The lyophilized powder may be reconstituted prior to administration in, e.g., water, normal saline, Ringer-Lactate solution, Ringer solution, or a buffered aqueous solution. The lyophilized powder can provide a longer stable shelf-life for the psilocin benzoate drug product.

The above-described compositions, in any of the forms described above, can be used for treating a disease or condition described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment. A pharmaceutical composition of this invention can be administered parenterally or intravenously following dissolution of the lyophilized powder. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

For use in the methods and compositions of the invention, the pharmaceutically acceptable psilocin salt may be contained in any appropriate amount in any suitable carrier substance formulated for intravenous infusion. Compositions for infusion use may be provided in unit dosage forms (e.g., in single-dose ampoules), or in vials containing several doses and in which a suitable preservative may be added.

The lyophilized solids may have a pH of between about 3.5 and about 5 (e.g., 3.5±0.5, 4±0.5, 4.5±0.5, or 5±0.5) when dissolve in between 10 mL and 50 mL (e.g., 10±5 mL, 15±5, 20±5, 25±5, 30±5, 35±5, 40±5, 45±5, or 50±5) of unbuffered water or in saline for injection. Furthermore, lyophilized powder may include a concentration of psilocin benzoate salt between about 0.01% (w/w) and about 10% (w/w) (e.g., 0.01% (w/w)±0.01% (w/w), 0.02% (w/w)±0.01% (w/w), 0.03% (w/w)±0.01% (w/w), 0.04% (w/w)±0.01% (w/w), 0.05% (w/w)±0.05% (w/w), 0.1% (w/w)±0.05% (w/w), 0.15% (w/w)±0.05% (w/w), 0.2% (w/w)±0.1% (w/w), 0.3% (w/w)±0.1% (w/w), 0.4 (w/w)±0.1% (w/w), 0.5% (w/w)±0.1% (w/w), 0.6% (w/w)±0.1% (w/w), 0.7 (w/w)±0.1% (w/w), 0.8 (w/w)±0.1% (w/w), 0.9 (w/w)±0.1% (w/w), 1% (w/w)±0.5% (w/w), 1.5% (w/w)±0.5% (w/w), 2% (w/w)±1% (w/w), 3% (w/w)±1% (w/w), 4% (w/w)±1% (w/w), 5% (w/w)±1% (w/w), 6% (w/w)±1% (w/w), 7% (w/w)±1% (w/w), 8% (w/w)±1% (w/w), 9% (w/w)±1% (w/w), or 10% (w/w)±1% (w/w)).

The lyophilized solid of the invention may include one or more bulking agents. The bulking agent may be a crystallizable bulking agent. Bulking agents typically are used in the art to provide structure and weight to the soli produced as a result of lyophilization. Any suitable bulking agent known in the art may be used in connection with the inventive lyophilized composition. Suitable bulking agents include, for example, mannitol, dextran, glycine, polyvvinylpyrrolidone (PVP), starch paste, pregelatinized starch, hydroxypropyl methyl cellulose (HPMC), carboxymethyl cellulose (CMC), or polyethylene glycol (PEG). In some embodiments, the bulking agent that is used is mannitol. The mannitol may be used to increase the critical temperature of the frozen formulation during the freeze-drying process. Mannitol typically forms a stable crystalline structure which provides a “scaffold” for the other components of the formulation which are likely to remain within the amorphous phase during freeze-drying. The crystalline mannitol has a high eutectic melting point and would thus enable the use of aggressive conditions during freeze-drying.

Without wishing to be bound by theory, freeze-drying the formulations described herein at temperatures above the critical temperature will likely cause some collapse or shrinkage within the final product cake. This may lead to increased residual moisture content and a low dry-state glass transition temperature, and may result in a final product with poor physicochemical and thermostability characteristics that limit its suitability for storage at higher temperatures. Therefore, to enable the freeze-drying to be undertaken more rapidly without compromising final product quality, it may be necessary to include a “collapse temperature modifier”, which will increase the critical temperature of the formulation. Such excipients exhibit a high collapse temperature and confer good mechanical strength to the final dried cake. The chosen collapse temperature modifier will help to ensure that appearance, shelf life, reconstitution characteristics and mechanical properties of the final product meet the required standard, whilst ensuring the process is efficient.

The bulking agent may be present in the lyophilized solid in an amount of between 65% (w/w) and 95% (w/w) (e.g., 65% (w/w)±5% (w/w), 70% (w/w)±5% (w/w), 75% (w/w)±5% (w/w), 80% (w/w)±5% (w/w), 85% (w/w)±5% (w/w), 90% (w/w)±5% (w/w), or 95% (w/w)±5% (w/w)). For example, the bulking agent may have a concentration in the lyophilized solid of between about 70% (w/w) and 90% (w/w) (e.g., 70% (w/w)±1% (w/w), 71% (w/w)±1% (w/w), 72% (w/w)±1% (w/w), 73% (w/w)±1% (w/w), 74% (w/w)±1% (w/w), 75% (w/w)±1% (w/w), 76% (w/w)±1% (w/w), 77% (w/w)±1% (w/w), 78% (w/w)±1% (w/w), 79% (w/w)±1% (w/w), 80% (w/w)±1% (w/w), 81% (w/w)±1% (w/w), 82% (w/w)±1% (w/w), 83% (w/w)±1% (w/w), 84% (w/w)±1% (w/w), 85% (w/w)±1% (w/w), 86% (w/w)±1% (w/w), 87% (w/w)±1% (w/w), 88% (w/w)±1% (w/w), 89% (w/w)±1% (w/w), or 90% (w/w)±1% (w/w)). The lyophilized solid of the invention can include one or more solvents, diluents, or other liquid vehicle, surface active agents, tonicity agents, and/or preservatives, as suited to the particular dosage form desired. Remington's Pharmaceutical Sciences, 23^{rd} Edition, E. W. Martin (Mack Publishing Co., Easton, Pa., 1990) discloses various excipients used in formulating pharmaceutical compositions and known techniques for the preparation thereof. Some examples of materials that can serve as pharmaceutically acceptable stabilizing excipients include, but are not limited to, sugars such as lactose, glucose, trehalose and sucrose; amino acids such as glycine or arginine for stabilization during freezing-drying; cellulose and its derivatives such as sodium carboxymethyl cellulose, ethyl cellulose and cellulose acetate; buffering agents such as citrate, acetate, and/or phosphate; pyrogen-free water; isotonic saline; Ringer's solution; 5% dextrose solution and combinations with the foregoing aqueous solutions. In some embodiments, the buffering agent is a citrate buffer. The buffering agent may be present in the lyophilized solid in an amount of less than 80% (w/w). wherein the solid comprises between 10% (w/w) and 95% (w/w) (e.g., 10% (w/w)±5% (w/w), 15% (w/w)±5% (w/w), 20% (w/w)±5% (w/w), 25% (w/w)±5% (w/w), 30% (w/w)±5% (w/w), 35% (w/w)±5% (w/w), 40% (w/w)±5% (w/w), 45% (w/w)±5% (w/w), 50% (w/w)±5% (w/w), 55% (w/w)±5% (w/w), 60% (w/w)±5% (w/w), 65% (w/w)±5% (w/w), 70% (w/w)±5% (w/w), 75% (w/w)±5% (w/w), 80% (w/w)±5% (w/w), 85% (w/w)±5% (w/w), 90% (w/w)±5% (w/w), or 95% (w/w)±5% (w/w)) buffering agent. For example, the lyophilized solid may include between 10% (w/w) and 30% (w/w) (e.g., 10% (w/w)±5% (w/w), 15% (w/w)±5% (w/w), 20 (w/w)±5% (w/w), 25% (w/w)±5% (w/w), or 30% (w/w)±5% (w/w)) buffering agent buffer of a buffering agent.

The lyophilized solid of the invention may include an antioxidant. An antioxidant may be used to improve the shelf-life stability of the psilocin benzoate. Antioxidants are known in the art. Non-limiting examples of an antioxidant include pyrroloquinoline quinone (PQQ), vitamin C, vitamin E (e.g., a tocopherol, such as α-tocopherol, β-tocopherol, or γ-tocopherol, or a mixture thereof), β-carotene, a polyphenol (e.g., a phenolic acid, a stilbene, or a flavonoid), or an inorganic selenium. In some embodiments, the antioxidant is selected from the group consisting of pyrroloquinoline quinone (PQQ), vitamin C, vitamin E, β-carotene, a polyphenol, thioglycerol, sodium bisulfite, carnosine, N-acetylcarnosine, pyruvate, astaxanthin, glutathione, cysteine, cysteine and combinations thereof. In some embodiments, the antioxidant is sodium bisulfite. The lyophilized solid may include less than 1% (w/w) of an antioxidant (e.g., about 0.01% (w/w), 0.1% (w/w), 0.2% (w/w), 0.3% (w/w), 0.4% (w/w), 0.5% (w/w), 0.6% (w/w), 0.7% (w/w), 0.8% (w/w), or 0.9% (w/w)). The lyophilized solid of the invention may be free of any antioxidant.

The lyophilized solid may include sodium chloride. For example, the solid may include less than 20% (w/w) by weight sodium chloride. In some embodiments, the lyophilized solid may be free of sodium chloride.

The above-described compositions, in any of the forms described above, may be stored in a light impenetrable container. For the example, the compositions described herein may be contained in an amber bottle or vial.

### Methods of Producing

The disclosure provides methods of producing the lyophilized solids described herein. The method includes treating an aqueous solution comprising the components of the lyophilized solid with a thermal treatment step and at least one drying step.

The thermal treatment may include multiple steps of treating the solution are various temperatures. For example, the thermal treatment step may include holding the solution at a temperature of between −50° C. and 10° C. for between 30 hours and 70 hours. In some embodiments, the thermal treatment step includes a first step, wherein the first step is performed at about 5° C. for about 10 minutes; a second step, wherein the second step is performed at about −40° C. for about 45 minutes; a third step, wherein the third step is performed at about −40° C. for about 120 minutes; a fourth step, wherein the fourth step is performed at about −9° C. for about 31 minutes; a fifth step, wherein the fifth step is performed at about −9° C. for about 300 minutes; a sixth step, wherein the sixth step is performed at about −40° C. for about 31 minutes; and a seventh step, wherein the seventh step is performed at about −40° C. for about 120 minutes. In some embodiments, the thermal treatment step may include fast freezing the solution by using an initial cooling rate of 1° C. per minute and subsequently performing an annealing step at −9° C.

The drying step may include between one and five steps. For example, the drying step may include a primary drying step and a secondary drying step. The primary drying step may be performed for between 5 hours and 10 hours. Furthermore, the primary drying step may be performed at a temperature of between −50° C. and 0° C. Lastly, the primary drying step may be performed at a pressure of between 20 μBar and 300 μBar. In some embodiments, the primary drying step includes a first step, a second step, and a third step, wherein the first step is performed at about −40° C. for about 60 minutes with a pressure of about 50 μBar, wherein the second step is performed at about −9° C. for about 31 minutes with a pressure of about 200 μBar, and wherein the third step is performed at −9° C. for about 2566 minutes with a pressure of about 200 μBar. The drying step may include a secondary drying step. For example, the secondary drying step may include a first step and second step, wherein the first step is performed at 25° C. for about 78 minutes at a pressure of about 50 μBar and wherein the second step is performed at 25° C. for about 480 minutes at a pressure of about 50 μBar. There may be a third drying step occurring following the primary and secondary drying step. The third drying step may include drying at a temperature of between 15° C. and 50° C. for between 100 minutes and 200 minutes at a pressure of between 20 μBar and about 50 μBar. For example, the third drying step may include five steps, wherein the first step is performed at about 20° C. for about 5 minutes at a pressure of about 1 μBar, wherein the second step is performed at about 25° C. for about 5 minutes at a pressure of about 50 μBar, wherein the third step is performed at about 25° C. for about 15 minutes at a pressure of about 50 μBar, wherein the fourth step is performed at about 45° C. for about 20 minutes at a pressure of about 50 μBar, and wherein the fifth step is performed at about 45° C. for about 120 minutes at a pressure of about 50 μBar. Accordingly, the resulting lyophilized solid including psilocin benzoate may include less than 3% (w/w) (e.g., less than 1% (w/w)).

### Treatment Methods

The disclosure provides lyophilized solids comprising psilocin benzoate which may be dissolved in an aqueous solution such that it may be intravenously administered to treat psychological conditions, neurological injuries, pain, cephalic pain (e.g., headache), inflammatory conditions, and anxiety.

### Psychological Conditions

The psilocin benzoate formulations of the invention can be used to treat psychological conditions. The psychological condition may be any psychological condition described herein. In some embodiments the psychological condition is depression, anxiety, addiction, post-traumatic stress disorder (PTSD), an eating disorder, or compulsive behavior. In some embodiments, the psychological condition may be depression. The psychological condition may also be anxiety. The anxiety may be experienced by a subject who is receiving palliative care or is enrolled in a hospice program. In certain embodiments, the subject who is experiencing anxiety has symptoms such as hypervigilance, fatigue, racing thoughts, irritability, excessive worry, and/or fear.

The subject diagnosed with a psychological condition may be diagnosed by evaluation of the subject's symptoms by a physician, clinician, or therapist based on a physical examination. For example, a blood test may be used to evaluate blood concentration levels of certain biomarkers such as hormones, calcium, vitamin D, electrolytes, and iron in diagnosing depression. Additionally, or alternatively, for patients with a possible depression condition a depression screening test may be performed by the physician, clinician, or therapist to aid in the diagnosis of depression. In some embodiments, the methods described herein may be used to treat psychosomatic pain conditions. In some embodiments, the psychosomatic pain condition may be fibromyalgia, chronic fatigue, migraines, or back pain.

### Neurological Injuries

The psilocin benzoate formulations of the invention can be used to treat a neurological injury. The neurological injury may be any neurological injury. In some embodiments, the neurological injury is a stroke, a traumatic brain injury, or a spinal cord injury. The methods of treating a neurological injury described herein may reduce acute inflammation. In certain embodiments, hippocampal hyperactivity is reduced. In particular embodiments, the methods of the invention are used to treat a neurological injury, e.g., stroke, traumatic brain injury, and spinal cord injury, by administering intravenous infusions of psilocin benzoate with an antioxidant as needed for pain, inflammation, and/or other symptoms associated with the neurological injury.

### Neurodegenerative Conditions

The psilocin benzoate formulations of the invention can be used to treat neurodegenerative conditions. The neurodegenerative condition to be treated can be Alzheimer's disease, Huntington's disease, or Parkinson's disease, among others.

### Inflammatory Conditions

The psilocin benzoate formulations of the invention can be used to treat inflammatory conditions. The inflammatory condition to be treated can be a lung inflammation (e.g., chronic obstructive pulmonary disease (COPD)), neuroinflammation (e.g., inflammation associated with Alzheimer's disease), chronic inflammation, rheumatoid arthritis, atherosclerosis, psoriasis, type II diabetes, inflammatory bowel disease, Crohn's disease, multiple sclerosis, and/or septicemia.

### Chronic Pain

The psilocin benzoate formulations of the invention can be used to treat conditions associated with chronic pain. The chronic pain may result from post-operative pain, tension headaches, chronic lower back pain, fibromyalgia, nephropathy, multiple sclerosis, shingles, complex regional pain syndrome, cephalic pain, or sciatica. The chronic pain may arise from an operation. The chronic pain may also be pain associated with a particular disease or condition such as nephropathy, multiple sclerosis, shingles, or complex regional pain syndrome. As used herein, a disorder or condition associated with cephalic pain is a disorder or condition which has as one of its symptoms cephalic/head pain (e.g., headache). Examples of such disorders or conditions include trigeminal autonomic cephalalgias such as episodic and chronic cluster headache (CH), episodic and chronic paroxysmal hemicrania (PH), and short-lasting unilateral neuralgiform headache attacks with conjunctival injection and tearing (SUNCT). Other examples of disorders or conditions which can be treated according to the present invention include vascular headaches (e.g., migraine headaches), tension headaches, headaches associated with the use of a substance (e.g., triptans such as sumatriptan, benzodiazepines such as alprazolam, analgesics such as ibuprofen, ergots such as ergotamine, opioids such as morphine, recreational drugs such as caffeine, nicotine, alcohol, and hormone replacement therapy containing, for example, estrogen) or its withdrawal. Yet additional examples of disorders or conditions associated with cephalic pain include miscellaneous headache unassociated with a structural lesion, headache associated with a nonvascular intracranial disorder, headache associated with a non-cephalic infection, headache associated with a metabolic disorder, headache associated with a disorder of the cranium, neck, eyes, nose, sinuses, teeth, mouth, or other facial or cranial structure, nerve trunk pain and deafferentation pain.

### Items

1. A lyophilized solid comprising (i) an amorphous acid addition salt of psilocin benzoate; (ii) a buffering agent; and (iii) less than 2% (w/w) water.
2. The lyophilized solid of item 1, wherein the psilocin benzoate has a concentration of between 0.01% (w/w) and 10% (w/w).
3. The lyophilized solid of item 1 or 2, wherein the solid comprises less than 80% (w/w) of a buffering agent.
4. The lyophilized solid of item 3, wherein the solid comprises between 10% (w/w) and 95% (w/w) buffering agent.
5. The lyophilized solid of item 3, wherein the solid comprises between 10% (w/w) and 30% (w/w) buffering agent buffer.
6. The lyophilized solid of any one of items 1-5, wherein the buffering agent is a citrate buffer.
7. The lyophilized solid of any one of items 1-6, wherein further comprises a bulking agent.
8. The lyophilized solid of item 7, wherein the bulking agent has a concentration of between 65% (w/w) and 95% (w/w).
9. The lyophilized solid of item 8, wherein the bulking agent has a concentration of between 70% (w/w) and 90% (w/w).
10. The lyophilized solid of any one of items 7-9, wherein the bulking agent is mannitol.
11. A lyophilized solid comprising (i) an amorphous psilocin benzoate in an amount of between 0.01% (w/w) and 10% (w/w), (ii) a buffering agent in an amount of between 10% (w/w) and 30% (w/w); (iii) a bulking agent in an amount of between 70% (w/w) and 95% (w/w); and (iii) less than 3% (w/w) water.
12. The lyophilized solid of item 11, wherein the buffer is a citrate buffer.
13. The lyophilized solid of item 11 or 12, wherein the bulking agent comprises mannitol.
14. The lyophilized solid of any one of items 1-13, wherein the solid comprises less than 1% (w/w) water.
15. The lyophilized solid of any one of items 1-14, wherein the solid has a pH of between 3.5 and 5 when dissolved in between 10 mL and 50 mL of unbuffered water or saline for injection.
16. The lyophilized solid of any one of items 1-15, wherein the solid further comprises less than 1% (w/w) of an antioxidant.
17. The lyophilized solid of item 16, wherein the antioxidant is sodium bisulfite.
18. The lyophilized solid of any one of items 1-15, wherein the solid is free of any antioxidant.
19. The lyophilized solid of any one of items 1-18, wherein the solid comprises less than 20% (w/w) by weight sodium chloride.
20. The lyophilized solid of any one of items 1-18, wherein the solid is free of sodium chloride.
21. A method of preparing a lyophilized solid comprising (i) an amorphous acid addition salt of psilocin benzoate; (ii) a buffering agent; and (iii) less than 3% (w/w) water, wherein the solid of any one of items 1-20 is dissolved in between 5 mL to 50 mL of an aqueous solution and undergoes a thermal treatment step, followed by a drying step.
22. The method of item 21, wherein the thermal treatment step is performed for between 30 hours and 70 hours at a temperature of between −50° C. and 10° C.
23. The method of item 22, wherein the thermal treatment step comprises between 3 and 8 steps.
24. The method of item 23, wherein the thermal treatment step comprises:
   (i) a first step, wherein the first step is performed at about 5° C. for about 10 minutes;
   (ii) a second step, wherein the second step is performed at about −40° C. for about 45 minutes;
   (iii) a third step, wherein the third step is performed at about −40° C. for about 120 minutes;
   (iv) a fourth step, wherein the fourth step is performed at about −9° C. for about 31 minutes;
   (v) a fifth step, wherein the fifth step is performed at about −9° C. for about 300 minutes;
   (vi) a sixth step, wherein the sixth step is performed at about −40° C. for about 31 minutes; and
   (vii) a seventh step, wherein the seventh step is performed at about −40° C. for about 120 minutes.
25. The method of any one of items 21-24, wherein the drying step comprises a primary drying step and a secondary drying step.
26. The method of item 25, wherein the primary drying step is performed for between 5 hours and 10 hours at a temperature of between −50° C. and 0° C., and a pressure of between 20 μBar and 300 μBar.
27. The method of item 26, wherein the primary drying step comprises a first step, a second step, and a third step, wherein the first step is performed at about −40° C. for about 60 minutes with a pressure of about 50 μBar, wherein the second step is performed at about −9° C. for about 31 minutes with a pressure of about 200 μBar, and wherein the third step is performed at −9° C. for about 2566 minutes with a pressure of about 200 μBar.
28. The method of item 26 and 27, wherein the secondary dry step comprises a first step and second step, wherein the first step is performed at 25° C. for about 78 minutes at a pressure of about 50 μBar and wherein the second step is performed at 25° C. for about 480 minutes at a pressure of about 50 μBar.
29. A method of treating a disease or condition in a subject in need thereof, the method comprising intravenously administering to the subject a lyophilized solid of any one of items 1-20 dissolved in from 5 mL to 50 mL of an aqueous solution in an amount sufficient to treat the disease or condition.

### CLAUSES

1. A lyophilized solid comprising:
(i) an amorphous acid addition salt of psilocin benzoate;
(ii) a buffering agent; and
(iii) less than 2% (w/w) water.
2. The lyophilized solid of clause 1, wherein the psilocin benzoate has a concentration of between 0.01% (w/w) and 10% (w/w).
3. The lyophilized solid of clause 1 or 2, wherein the solid comprises less than 80% (w/w) of a buffering agent.
4. The lyophilized solid of clause 1 or clause 2, wherein the solid comprises between 10% (w/w) and 95% (w/w) or between 10% (w/w) and 30% (w/w) buffering agent.
5. The lyophilized solid of any one of clauses 1-4, wherein the buffering agent is a citrate buffer.
6. The lyophilized solid of any one of clauses 1-5, wherein the solid further comprises a bulking agent.
7. The lyophilized solid of clause 6, wherein the bulking agent has a concentration of between 65% (w/w) and 95% (w/w) or between 70% (w/w) and 90% (w/w).
8. The lyophilized solid of any one of clauses 6 or 7, wherein the bulking agent is mannitol.
9. A lyophilized solid comprising:
(i) an amorphous psilocin benzoate in an amount of between 0.01% (w/w) and 10% (w/w);
(ii) a buffering agent in an amount of between 10% (w/w) and 30% (w/w);
(iii) a bulking agent in an amount of between 70% (w/w) and 95% (w/w); and
(iii) less than 3% (w/w) water.
10. The lyophilized solid of clause 9, wherein the buffer is a citrate buffer and wherein the bulking agent comprises mannitol.
11. The lyophilized solid of any one of clauses 1-10, wherein the solid comprises less than 1% (w/w) water.
12. The lyophilized solid of any one of clauses 1-11, wherein the solid has a pH of between 3.5 and 5 when dissolved in between 10 mL and 50 mL of unbuffered water or saline for injection.
13. The lyophilized solid of any one of clauses 1-12, wherein the solid further comprises less than 1% (w/w) of an antioxidant.
14. The lyophilized solid of clause 13, wherein the antioxidant is sodium bisulfite.
15. The lyophilized solid of any one of clauses 1-12, wherein the solid is free of any antioxidant.
16. The lyophilized solid of any one of clauses 1-15, wherein the solid comprises less than 20% (w/w) by weight sodium chloride.
17. The lyophilized solid of any one of clauses 1-15, wherein the solid is free of sodium chloride.
18. A method of preparing a lyophilized solid comprising:
(i) an amorphous acid addition salt of psilocin benzoate;
(ii) a buffering agent; and
(iii) less than 3% (w/w) water,
wherein the solid of any one of clauses 1-17 is dissolved in between 5 mL to 50 mL of an aqueous solution and undergoes a thermal treatment step, followed by a drying step.
19. The method of clause 18, wherein the thermal treatment step is performed for between 30 hours and 70 hours at a temperature of between −50° C. and 10° C.
20. The method of clause 19, wherein the thermal treatment step comprises:
   (i) a first step, wherein the first step is performed at about 5° C. for about 10 minutes;
   (ii) a second step, wherein the second step is performed at about −40° C. for about 45 minutes;
   (iii) a third step, wherein the third step is performed at about −40° C. for about 120 minutes;
   (iv) a fourth step, wherein the fourth step is performed at about −9° C. for about 31 minutes;
   (v) a fifth step, wherein the fifth step is performed at about −9° C. for about 300 minutes;
   (vi) a sixth step, wherein the sixth step is performed at about −40° C. for about 31 minutes; and
   (vii) a seventh step, wherein the seventh step is performed at about −40° C. for about 120 minutes.
21. The method of any one of clauses 18-20, wherein the drying step comprises a primary drying step and a secondary drying step.
22. The method of clause 21, wherein the primary drying step is performed for between 5 hours and 10 hours at a temperature of between −50° C. and 0° C., and a pressure of between 20 μBar and 300 μBar.
23. The method of clause 22, wherein the primary drying step comprises a first step, a second step, and a third step, wherein the first step is performed at about −40° C. for about 60 minutes with a pressure of about 50 μBar, wherein the second step is performed at about −9° C. for about 31 minutes with a pressure of about 200 μBar, and wherein the third step is performed at −9° C. for about 2566 minutes with a pressure of about 200 μBar.
24. The method of clause 22 and 23, wherein the secondary dry step comprises a first step and second step, wherein the first step is performed at 25° C. for about 78 minutes at a pressure of about 50 μBar and wherein the second step is performed at 25° C. for about 480 minutes at a pressure of about 50 μBar.
25. A lyophilized solid of any one of clauses 1-17 dissolved in from 5 mL to 50 mL of an aqueous solution for use in a method of treating a disease or condition in a subject in need thereof, the method comprising intravenously administering to the subject the lyophilized solid dissolved in from 5 mL to 50 mL of an aqueous solution in an amount sufficient to treat the disease or condition.

### EXAMPLES

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1. Formulation Optimization

The goal of this study was to develop an isotonic stable formulation with the psilocin benzoate Drug Substance (DS) concentration ranging from 0.5 to 3.0 mg/mL (mg free base psilocin) as a bulk sterile solution, having a pH in the range of 4 to 9 in a single use glass vial suitable for IV injection. However, DS was shown to have a short stability in aqueous solution at the desired pH range (rapid degradation and discoloration over a short period of time). This led to the development of a formulation of psilocin benzoate containing antioxidants, buffers, pH adjustment.

The psilocin benzoate drug product (DP) powder for solution for infusion was formulated as a sterile lyophilized powder for reconstitution in single use 6 mL glass vials. The DP was supplied in dose corresponding to 1.0 to 6.0 mg of the DS as a free base with standard compendial excipients commonly used in the pharmaceutical industry for parenteral injection.

Sterile commercially available normal saline for injection, 0.9% NaCl, was used as the diluent for the preparation of the final reconstituted solution for infusion at a final volume of 30 mL/syringe.

The attributes and target profile of the formulation are detailed in Table 1.

TABLE 1
Attributes and Target Product Profile of the formulation

| Attributes | Target Product Profile |
|---|---|
| Dosage Form and Strength | A lyophilized powder for solution in single use, sterile glass vials |
| | 6 mg/vial Psilocin Benzoate as a free base to support a range of |
| | clinical doses |
| Formulation | Isotonic stable, sterile formulation with a pH in the range of 4 to 5 in |
| | a single use glass vial with standard compendial excipients |
| | commonly used in the pharmaceutical industry for parenteral |
| | infusion. |
| Fill volume | 2 ml/vial and final reconstituted solution for infusion at a final volume |
| | of 30 mL/syringe. |
| Container | Type I glass vials (DIN 6R) with 20 mm rubber stoppers and sealed |
| | with 20 mm aluminum caps. |
| Appearance | White, elegant looking final dried cakes with no signs of collapse or |
| | shrinkage |
| | Clear, colorless solution after reconstitution |
| Shelf Life | Stable for at least 2 years |
| | Ambient (ideally) or 2-8° C. storage conditions |
| | Glass transition temperature from solid state mDSC thermal analysis |
| | can guide the storage condition |
| Residual Moisture | Final cake residual moisture of <2% w/w (KF analysis) |
| Assay /impurity profile | Final stability of the DS to be assessed by HPLC assay/ impurity |
| | profile performance |
| Reconstitution characteristics | <60 sec dissolves completely without mixing leaving no visible |
| | residues as undissolved matter. |
| Mechanical Properties | Structurally robust white freeze-dried cakes |

### Initial Formulation Development

The initial formulation was formulated for intravenous infusion for use in small scale, with sufficient short-term stability (maximum 6 months).

The initial goal for early phase clinical trials was to develop an iso-osmotic formulation at required concentration range, having a pH in the range of 4 to 9, which is suitable for intravenous infusion.

Previous studies indicated that the DS (Psilocin benzoate salt) in solution had limited physical and chemical stability. Consequently, the following principal approaches, intended to maximize the stability, were assessed as part of the formulation development: pH adjustment, use of buffers and antioxidants.

To minimize oxidation, numerous antioxidants at several concentrations were tested, it was found that 0.01% w/v sodium bisulfite gave optimal results to maximize the physical and chemical stability of the formulation and prevent discoloration.

Preliminary pH stability experiments were conducted with a few conventional buffers aiming to identify the most suitable candidates and provide optimal pH stability profile for formulating DP. Optimal stability was observed to improve under slightly acidic conditions and the optimal pH was achieved using the 75 mM citrate buffer at nominal pH 4.5.

The use of an antioxidant excipient, optimal pH, exclusion of the formulation from light and longer term refrigerated storage improved stability in solution.

A 75 mM citrate buffer in 0.4% w/v sodium chloride, 0.01% w/v sodium bisulfite, pH 4.5 was chosen as the formulation which was proven to be superior with respect to all the targeted DP characteristics. The citrate buffer at pH 4.5 was prepared in 0.40% w/v sodium chloride solution to increase the tonicity of the formulation, and sodium bisulfite was added as an antioxidant.

The intended final volume is up to 30 mL with a dose range between a minimum of 0.25 mg and a maximum of 6.0 mg of the DS, as a free base. In terms of concentration this was equivalent to 16.67 μg/mL to 200.0 μg/mL, expressed as free base.

### Lyophilized Powder Formulation

A lyophilized powder for solution for infusion was developed with an optimized formulation to improve stability potential through freeze-dry cycle and increase long-term shelf life. The composition of the final formulation is based off stability results of earlier pH and formulation screening studies conducted at early stage of development. Data generated during these studies suggests that citrate buffer in the pH range of 4 to 5 exhibits the best solution stability (pre-lyophilization) prior to freeze drying.

The lyophilized powder formulation composition was optimized to remove the sodium bisulfite antioxidant, decreased significantly the concentration of the citrate buffer and add mannitol as bulking agent that will be suitable for freeze-drying and to establish a suitable drying recipe for the selected formulation. This was achieved by conducting four freeze-drying cycles, each with accompanying pre and post-lyophilization analysis of the resulting products;

Freeze-drying microscopy (FDM) analysis of proposed candidate formulations (CFs) was performed to help establish preliminary conditions for the freeze-drying cycle and for subsequent optimization cycles. Modulated Differential Scanning Calorimetry (MDSC) analysis and appearance analysis of the resulting freeze-dried products was also performed. Additionally, two optimization cycles were performed in which the conditions used during the thermal treatment, primary drying, and secondary drying phases of the process were adjusted to establish an efficient and robust recipe for freeze-drying of the selected CFs.

A final assessment cycle was conducted using the established cycle conditions to yield a final product for subsequent stability studies. A post-lyophilization analytical analysis of the freeze-dried products was obtained from each cycle to assess the product appearance, physicochemical characteristics, and thermostability characteristics.

The buffering components of the formulation were a citrate buffer consisting of both citric acid monohydrate and tri-sodium citrate at concentrations calculated to achieve a final citrate concentration of 25 mM and a pH of 4.5. Additionally, due to the susceptibility of the DS to oxidation whilst in aqueous solution, the formulation initially also included 0.01% sodium bisulphite as an antioxidant.

To improve the suitability of the existing formulation for the freeze-drying process, it was necessary to include additional excipients that would serve as a cryoprotectant and/or lyoprotectant. These could serve to protect the activity of the API during freeze-drying and subsequent storage and could improve the physicochemical and thermostability characteristics of the formulation. A number of excipients potentially suitable for use in the freeze-dried formulation were evaluated.

Two presentations of each formulation, containing either a 6 mg dose of psilocin benzoate (designated presentation “A”) or a 1 mg dose of the API (designated presentation “B”) were investigated. The compositions of the formulations that were screened in the first cycle are provided in Table 2 below.

All proposed excipient combinations contained either mannitol or dextran, which both served the purpose of increasing the critical temperature of the frozen formulation during the freeze-drying process. Mannitol was selected as a potential excipient because crystalline mannitol has a high eutectic melting point and would thus enable the use of aggressive conditions during freeze-drying. Dextran was selected as a potential excipient since it typically remains amorphous during freeze-drying and increases the collapse temperature of the amorphous phase substantially, enabling the use of aggressive conditions during freeze-drying. CFs 3, 4 and 5, also included disaccharides (e.g., lactose and/or sucrose) as stabilizers.

TABLE 2
Summary of the Compositions of Solutions used in the Initial Cycle

| | Excipient 1 | Excipient 2 | Psilocin Benzoate | |
|---|---|---|---|---|
| CF | (Conc) | (Conc) | conc (mg/ml) | Buffer/ Antioxidant |
| 01A | Mannitol (4%) | | 3 | 9.55 mg/mL Citric Acid |
| 01B | | | 0.5 | 7.45 mg/mL Trisodium |
| 02A | Dextran (4%) | | 3 | Citrate |
| 02B | | | 0.5 | 0.01% Sodium |
| 03A | Mannitol (3%) | Lactose (1%) | 3 | Bisulphite |
| 03B | | | 0.5 | |
| 04A | Mannitol (3%) | Sucrose (1%) | 3 | |
| 04B | | | 0.5 | |
| 05A | Dextran (3%) | Lactose (1%) | 3 | |
| 05B | | | 0.5 | |

A 25 mM citrate buffer at pH 4.5, 3% w/v mannitol was developed as the lyophilized formulation, where the lyophilized powder bulk vial was reconstituted and diluted in 0.9% w/v sodium chloride solution resulting in an aqueous isotonic solution for infusion.

The parameters of the freeze-drying cycle were established based on the critical temperatures obtained from FDM (freeze-drying microscopy) analysis of candidate formulations. The aim of this optimization cycle was to demonstrate that one or more of the selected candidate formulations are suitable as freeze-dried products and to establish preliminary pressure and temperature conditions necessary to achieve a product temperature suitable for yielding stable freeze-dried cakes for candidate formulation.

A comparison of original and revised solutions for infusion (30 mL/syringe) is shown in Table 3.

TABLE 3
Comparison of Original and Revised Formulations

| Component | Original Formulation | Revised Formulation |
|---|---|---|
| Psilocin benzoate, free base | 16.67 to 200.0 μg/mL | 33.33 to 200.0 μg/mL |
| Mannitol | — | 0.2% w/v (60.0 mg) |
| Citric Acid Monohydrate | 0.69% w/v (208.6 mg) | 0.02% w/v (5.2 mg) |
| Sodium citrate dihydrate | 1.24% w/v (371.3 mg) | 0.03% w/v (7.4 mg) |
| Sodium Bisulfite | 0.01% w/v (3.0 mg) | — |
| 1M Sodium hydroxide/Hydrochloric acid | — | — |
| pH (range) | 4.5 (3.5-5.0) | 4.5 (3.5-5.0) |
| Water for Injection (WFI) | q.s. | q.s. |
| Sodium chloride (NaCl) | 0.40% w/v (120.0 mg) | 0.90% w/v (270.0 mg) |

WFI- used as the process solvent, removed during freeze-drying cycle. q.s: quantity sufficient -quantity added up to total fill volume.
NaCl 0.9% w/v sterile saline for injection serves as diluent for the preparation of the final reconstituted/diluted solution for infusion

### Lyophilization Cycle Development

The formulations described in Table 1 above were prepared in accordance with the approved protocol. Prior to conducting the freeze-drying cycle, candidate formulations 1 and 5 were analyzed by Freeze-Drying Microscopy (FDM) to ascertain the upper control limit for the product temperature during the primary drying phase and provide a guide for the selection of the shelf temperature and chamber pressure setpoints during the primary drying phase. The collapse temperatures were established, as given in Table 4.

TABLE 4
Summary of observations made in BTL Lyostat analysis

| Formulation | Collapse (onset temperature) |
|---|---|
| CF 01, Mannitol (4%) | −38.8° C. |
| CF 05, Dextran (3%) & Lactose (1%) | −20.7° C. |

The shelf temperature and chamber pressure setpoints used during the primary drying phase were selected to achieve a target product temperature between −40.8° C. to −45.8° C. Whilst the FDM analysis demonstrated that a higher critical temperature could be achieved after the inclusion of an annealing step, it was unclear whether the annealing step employed would be adequate to ensure even crystallinity of the mannitol across the whole batch, and therefore, the conditions employed were deliberately conservative to ensure stability of all vials. An annealing step of −25° C. was typically set for three hours, along with a standard secondary drying step of 8 hours. The product probe temperature increased and converged with the shelf temperature after approximately 3500 minutes of primary drying. However, since product probes are typically non-representative of the batch, and because deliberately conservative conditions were being employed, a lengthy soak period of approximately 3000 minutes was applied. Upon completion of the freeze-drying cycle, the vials were backfilled and stoppered before being unloaded and crimped. Upon inspection of the appearance, all samples obtained from mannitol based formulations 1, 3 and 4 were given a score of 5 from 5, which corresponds to the highest level of acceptable freeze-dried product appearance in that it is white, elegant looking final dried cakes with no signs of collapse or shrinkage. The samples showed no signs of collapse but exhibited slight shrinkage in lactose added formulations. These formulations exhibited similar thermal behavior, with each undergoing a glass transition in the region of ~43° C. to ~57° C. A summary of these analytical results is provided in Table 5.

TABLE 5
Summary of the analytical results of formulations over time

| | Acceptance | Initial time | | 2-week time point | | | | | |
|---|---|---|---|---|---|---|---|---|---|
| Test | Criteria | point | | 2-8° C. | | 25° C./60% RH | | 40° C./75% RH | |
| Appearance | White | conforms | | conforms | | conforms | | conforms | |
| | lyophilized | | | | | | | | |
| | powder in a | | | | | | | | |
| | 6 mL glass | | | | | | | | |
| | vial. | | | | | | | | |
| Appearance | A clear, | A clear, | | A clear, colorless | | A clear, colorless | | A clear, colorless | |
| of | colorless | colorless | | solution | | solution | | solution | |
| reconstituted | liquid, free | solution | | | | | | | |
| solution and | of visible | | | | | | | | |
| visible | particles | | | | | | | | |
| particles | | | | | | | | | |
| Completeness | Dissolves | conforms | | conforms | | conforms | | conforms | |
| of | completely | | | | | | | | |
| reconstituted | in 2 mL | | | | | | | | |
| solution and | diluent | | | | | | | | |
| dissolution | within 60 | | | | | | | | |
| time | sec | | | | | | | | |
| pH of | 3.5-5.0 | 4.2 | | 4.2 | | 4.2 | | 4.2 | |
| reconstituted | | | | | | | | | |
| solution | | | | | | | | | |
| Water content | NMT 2.0%. | 2.5 | | — | | 2.0 | | 2.8 | |
| (Karl Fisher) | | | | | | | | | |
| Assay | 90.0%- | 97.8% | | 98.9% | | 98.4% | | 95.1% | |
| (labeled | 110.0% | | | | | | | | |
| amount) | | | | | | | | | |
| | | RRT | % Area | RRT | % Area | RRT | % Area | RRT | % Area |
| Related | NMT 1.0% | 1.80 | 0.30 | 1.05 | 0.16 | 0.88 | 0.17 | 0.88 | 0.16 |
| Substances | Total: NMT | Total | 0.30 | 1.62 | 0.15 | 1.05 | 0.17 | 1.05 | 0.18 |
| Unspecified | 5.0% | % Area | | 1.80 | 0.28 | 1.62 | 0.18 | 1.62 | 0.19 |
| impurities, | | | | Total | 0.59 | 1.80 | 0.29 | 1.80 | 0.29 |
| each | | | | % Area | | Total | 0.81 | Total | 0.82 |
| Total | | | | | | % Area | | % Area | |
| impurities | | | | | | | | | |

### Optimization Cycle E

Based on the results obtained from the initial cycle it was decided that dextran and mannitol formulations would be brought forward for further investigation and the excipient content was reduced from 4% to 3%. The candidate formulations were prepared as per the procedure outlined in the approved protocol and a sample of each was subject to FDM analysis. A summary of the formulation compositions and observed collapse temperatures is provided in Table 6.

TABLE 6
Summary of the Compositions of Proposed CF

| | | Excipient 1 | | Collapse onset |
|---|---|---|---|---|
| CF | Formulation | (Conc) | API/Buffer/Antioxidant | temperature |
| 01 | 1 | Mannitol | Psilocin Benzoate: 3 mg/mL(6 mg/2 | −33.3° C. |
| | | (3%) | ml vial); 8.14 mg/mL Citric Acid; 9.6 | |
| 02 | 2 | Dextran | mg/mL Trisodium Citrate; 0.01% | −20.4° C. |
| | | (3%) | Sodium Bisulphite; pH 4.4 | |

The aim of this cycle was to further optimize the freeze-drying recipe for the candidate formulations brought forward. The appearance of mannitol-based formulations obtained from the first cycle demonstrated that the annealing step employed was adequate to ensure uniform mannitol crystallinity, indicating that a significantly higher product temperature could be employed. The shelf temperature and chamber pressure setpoints were, therefore, increased from −30° C. to −20° C. and from 100 μBar to 200 μBar, respectively. All other cycle parameters were kept the same. The use of more aggressive conditions enabled a total reduction in cycle length of ~66 hours. According to the data obtained from this cycle and the associated post-lyophilization analysis, both candidate formulations represented a suitable formulation for freeze-drying. However, the dextran formulation had shrinkage away from the sides of the vial.

The post-lyophilization analysis conducted on samples obtained from the mannitol formulation revealed that the residual moisture was unexpectedly high (in the range of −4%). It was believed that the high residual moisture content was a result of either micro-collapse of the amorphous phase of the frozen material, masked by the crystalline mannitol present within the formulation, or that the mannitol had adopted a hemihydrate conformation that incorporated water molecules within the crystal lattice and was, thus, harboring a significant amount of water. The presence of either micro-collapse or mannitol hemihydrate were both undesirable, since either can lead to instability of the product during storage and, therefore, it was important to undertake an additional optimization cycle that incorporated measures to avoid these phenomena. To confirm whether mannitol hemihydrate or micro-collapse was the root cause, XRPD analysis of the samples from the first optimization cycle was conducted. This analysis revealed that mannitol hemihydrate was present within the samples, indicating that this was likely the source of the increased residual moisture. This was coupled with the appearance of endothermic events in the non-reversing heat flow signals of the MDSC analysis thermograms, which may have been indicative of mannitol hemihydrate (MHH) dehydration. A summary of the analytical results is provided in Table 7.

TABLE 7
Summary of the analytical results of formulations over time

| | Acceptance | | | 4-week time point | |
|---|---|---|---|---|---|
| Test | Criteria | Initial time point | | at 40° C./75% RH | |
| Appearance | White lyophilized | conforms | | conforms | |
| | powder in a 6 mL | | | | |
| | glass vial. | | | | |
| Appearance of | A clear, colorless | A clear, colorless solution | | A clear, colorless solution | |
| reconstituted solution and | liquid, free of | | | | |
| visible particles | visible particles | | | | |
| Completeness | Dissolves | conforms | | conforms | |
| of reconstituted | completely in 2 | | | | |
| solution and | mL diluent within | | | | |
| dissolution time | 60 sec | | | | |
| pH of | 3.5-5.0 | 4.3 | | 4.3 | |
| reconstituted solution | | | | | |
| Water content | NMT 2.0%. | 4.7% | | — | |
| (Karl Fisher) | | | | | |
| Direct addition | | | | | |
| KF | | | | | |
| Water content | | 3.8% | | 3.9% | |
| Oven-KF | | | | | |
| Assay | 90.0%-110.0% | 100.0% | | 98.0% | |
| | | RRT | % Area | RRT | % Area |
| Related | NMT 1.0% | 0.86 | 0.13 | 0.86 | 0.11 |
| Substances | | 1.38 | 0.14 | 1.37 | 0.15 |
| Unspecified | | 1.57 | 0.11 | 1.47 | 0.23 |
| impurities, each | Total: NMT 5.0% | 1.85 | 0.19 | 1.58 | 0.15 |
| Total | | 1.97 | 0.10 | 1.72 | 0.11 |
| impurities | | Impurities | 0.67 | 1.86 | 0.23 |
| | | Total % Area | | 1.97 | 0.11 |
| | | | | Impurities Total | 1.08 |
| | | | | % Area | |

The appearance, pH, and reconstitution time results confirmed that all criteria had been met for both formulations. The water content result was found to be significantly high in the mannitol formulation (4.7% by direct addition KF method and 3.8% by Oven-KF new optimized method). The assay and related substances results showed that the assay/impurities were affected at 40° C./75% RH, up to 4 weeks storage; where the concentration detected in the assay decreased from 100% to 98% and increased in impurities from 0.67% to 1.08%, after up to 4 weeks storage.

The presence of the high water content at the initial time point was shown to lead to potential instability of the product during storage since hydrolysis may be the main route of degradation. Additionally, low water content was desirable as the dried powder form with a low water content is very unlikely to support microbial growth

### Optimization Cycle 2

Despite showing promising results following optimization cycle 1, CF2 could not be progressed due to unfeasible regulatory requirements for dextran. Before progressing to the optimization cycle 2 with the CF1 mannitol formulation, it was necessary to investigate the root cause of the high residual moisture content. Powder X-Ray Diffraction (XRPD) analysis of a CF1 sample from optimization cycle 1 confirmed the presence of MHH in the freeze-dried product. To mitigate this, changes to the cycle parameters were introduced. Firstly, the thermal treatment stage of the recipe was altered to encourage mannitol crystallisation at >−10° C., by increasing the initial cooling rate from 0.5° C. to 1° C. per minute (to inhibit mannitol crystallisation on freezing) and subsequently performing an annealing step at −9° C. to encourage mannitol crystallisation at this temperature. Additionally, the formulations were split into two populations, one of which was subjected to an “extended” secondary drying regimen (heated at +25° C. for 8 hours, then heated at +45° C. for 2 hours), which has been shown to encourage the transition of MHH to anhydrous mannitol polymorphs. It was also decided that CF3 from the initial cycle would be re-introduced to provide a “backup”, should the measures taken to eliminate MHH from the final product prove ineffective. Changes to the concentration of buffering components were also made and the antioxidant sodium bisulphite was not included in the formulations on this occasion. A summary of the compositions of each of the candidate formulations investigated is provided in Table 8.

TABLE 8
Compositions of CFs Investigated

| | 2° D | Excipient | Excipient | Psilocin | |
|---|---|---|---|---|---|
| CF | Regimen | 1 (Conc) | 2 (Conc) | Benzoate | Buffer |
| 1A1 | Standard | Mannitol | | 3 mg/mL | 2.317 mg/mL |
| | | (3%) | | | Citric Acid; |
| 1A2 | Extended | | | | 4.125 mg/mL |
| 3A1 | Standard | | Lactose | | Trisodium |
| | | | (1%) | | Citrate; |
| 3A2 | Extended | | | | pH 4.5 |

### Optimization Cycle 2

The primary aim of this cycle was to further optimize the freeze-drying recipe for the candidate formulations selected without compromising product quality. Despite showing promising results following optimization cycle 1, CF2 could not be progressed due to unfeasible regulatory requirements for dextran. Before progressing to optimization cycle 2 with CF1 mannitol formulation, it was necessary to investigate the root cause of the high residual moisture content. Powder X-Ray Diffraction (XRPD) analysis of a CF1 sample from optimization cycle 1 confirmed the presence of MHH in the freeze-dried product. To mitigate this, changes to the cycle parameters were introduced.

In the second optimization cycle firstly, the cycle recipe was adjusted to include a faster freezing rate of 1° C. per minute down to the initial freezing point of −40° C. The thermal treatment stage of the recipe was altered to encourage mannitol crystallisation at >−10° C., by increasing the initial cooling rate from 0.5° C. to 1° C. per minute (to inhibit mannitol crystallisation on freezing). The use of a faster freezing rate helped to avoid mannitol crystallisation on freezing. This was important as the temperature at which mannitol crystallisation occurred could impact the conformation that is adopted. Subsequently, the temperature at which the frozen material was annealed was also increased from −25° C. to −9° C. to encourage mannitol crystallisation at this temperature. These measures were incorporated to prevent the formation of mannitol hemihydrate during the thermal treatment phase of the freeze-drying cycle. Additionally, the formulations were split into two populations, one of which was subjected to an “extended” secondary drying regimen (heated at +25° C. for 8 hours, then heated at +45° C. for 2 hours), which has been shown to encourage the transition of MHH to anhydrous mannitol polymorphs.

In addition to the above measures for preventing and/or eliminating mannitol hemihydrate, the second optimization cycle also included a second formulation which included 1% lactose as an excipient in addition to the 3% mannitol. This formulation was designed to help ascertain whether the micro-collapse was playing a role in the higher moisture content previously observed and would also serve a potential other option to the 3% mannitol formulation. Changes to the concentration of buffering components were also made in order to adjust some of the parameters of the formulation and the antioxidant sodium bisulphite was not included in the formulations on this occasion. A summary of the compositions of each of the candidate formulations investigated is provided in Table 9.

TABLE 9
Compositions of CFs Investigated in Optimisation Cycle 2

| | 2° D | Excipient | Excipient | Psilocin | |
|---|---|---|---|---|---|
| CF | Regimen | 1 (Conc) | 2 (Conc) | Benzoate | Buffer |
| CF 1A1 | Standard | Mannitol | | 3 mg/mL | 2.317 mg/mL |
| | | (3%) | | | Citric Acid; |
| CF 1A2 | Extended | | | | 4.125 mg/mL |
| CF 3A1 | Standard | | Lactose | | Trisodium |
| | | | (1%) | | Citrate; |
| CF 3A2 | Extended | | | | pH 4.5 |

The post-lyophilization analysis conducted on samples obtained from the second optimization cycle revealed that the use of a faster freezing rate and higher annealing temperature was sufficient to prevent the formation of mannitol hemihydrate. The residual moisture observed for formulation CF1A1 (mannitol based) was significantly lower than in the previous cycle (below 1%) and the thermogram obtained from Modulated Differential Scanning Calorimetry (MDSC) analysis did not exhibit any endothermic events in the non-reversing heat flow signal indicative of dehydration. These data therefore indicated that the mannitol hemihydrate was not present in these samples. Similar results were also observed for CF1A2 (mannitol & lactose based), which had been subject to the extended secondary drying regimen.

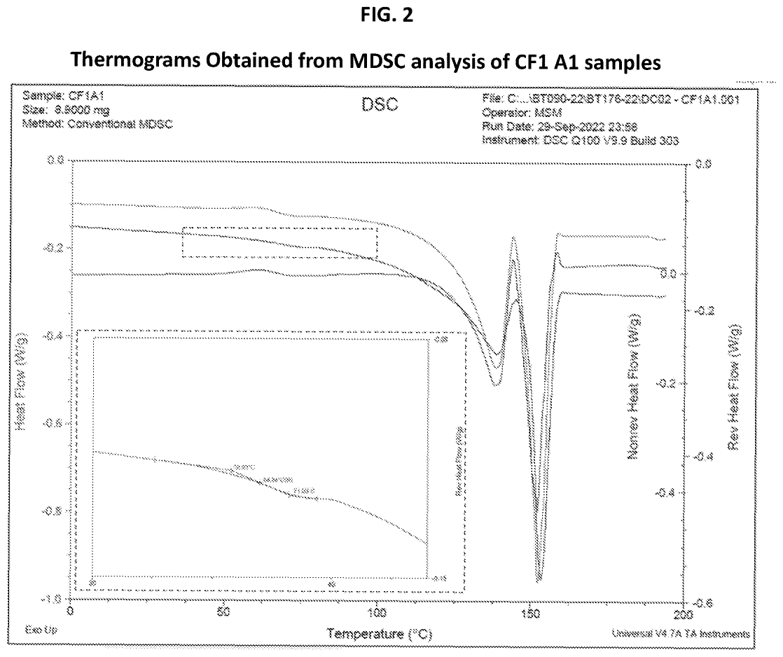

Thermograms of the raw data obtained from MDSC analysis of CF1A1, CF3A1, CF1A2 and CF3A2 are provided in FIGS. 2-4. A summary of the major events seen during each analysis is given in table 16.

The result of post-lyophilization thermal analysis and moisture analysis are provided in Table 10.

TABLE 10
Summary of MDSC thermal analysis and Karl Fischer moisture analysis

| | 2° D | Excipient 1 | Excipient 2 | Glass Transition | Moisture |
|---|---|---|---|---|---|
| CF | Regimen | (Conc) | (Conc) | Temp (Midpoint) | content (%) |
| CF 1A1 | Standard | Mannitol | — | Tg at 64.84° C. | 0.91 |
| CF 3A1 | | (3%) | Lactose | Tg at 65.69° C. | 1.58 |
| | | | (1%) | | |
| CF 1A2 | Extended | | — | Tg at 64.07° C. | 0.64 |
| CF 3A2 | | | Lactose | Tg at 71.99° C. | 0.78 |
| | | | (1%) | | |

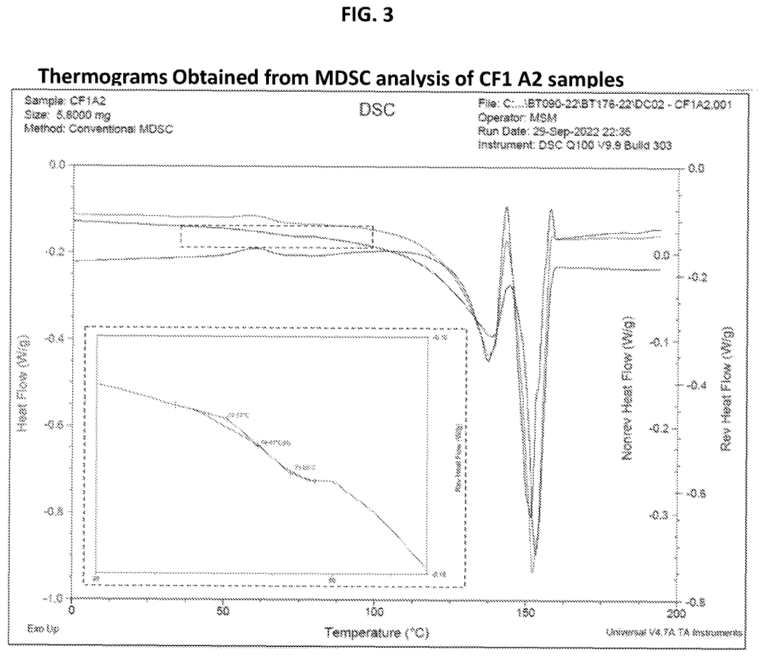

The results of the MDSC analysis indicate that the thermostability of all the freeze-dried cakes are very similar to one another. Thermograms of the raw data obtained from MDSC analysis of CF1A1 (mannitol only), CF3A1 (mannitol & lactose) are provided in FIGS. 2 and 3.

Each thermogram exhibited a glass transition (Tg) of the range of ~58° C. to ~75° C. Notably for the samples of CF1A1 and CF1A2 (mannitol only), no endothermic transitions were observed in the non-reversing heat flow signals, suggesting that MHH was not present in these samples. Whilst the thermograms for CF3A1 and CF3A2 (mannitol & lactose) did exhibit small endothermic transitions, these appeared to be substantially less pronounced than those observed from thermal analysis conducted following optimization cycle 1, suggesting that the proportion of MHH present is significantly reduced. As would be expected, the application of an extended secondary drying step at +45° C. resulted in a lower residual moisture content for both CF1A2 and CF3A2, compared with those subjected to standard secondary drying. The low residual moisture content observed for CF1A1 suggests that the use of a fast cooling rate and higher annealing temperature is sufficient to prevent that formation of MHH and that the use of the extended secondary drying step would be unnecessary for CF1. Whilst the residual moisture content of CF3A2 was also very low, a greater difference was observed with the moisture content for CF3A1, suggesting that the faster cooling rate and higher annealing temperature was less effective at preventing MHH formation in this formulation. The product temperature probe and pressure gauge data obtained during the lyophilization cycle appeared to indicate that further reduction of the primary drying time may still be achieved.

Two candidate formulations were each prepared in accordance with protocol above and freeze-dried. The total freeze-drying time was ~63 hours. The CF1A1 and CF3A1 vials were immediately crimped (standard), whilst the CF1A2 and CF3A2 vials (extended) subjected to the additional secondary drying step after completion of the main recipe. This additional secondary drying included a period of ~2 hours at which the product was held at +45° C.

The drying steps are summarized in Table 12 below.

Upon completion of the cycle all tubes were backfilled ~500 mBar with dry nitrogen gas and then crimped and transferred to the fridge for storage at 2-8° C. Photographs of a selection of product vials from each of the active formulations were taken.

### Results of Appearance Analysis

Upon inspection, all cakes were given a score of 5. As is usually the case with freeze-dried products containing crystalline mannitol, the resulting crystalline scaffold expands slightly to fill all the space occupied by the liquid formulation, generally resulting in a cosmetically acceptable freeze-dried product.

The pre-lyophilized solutions of each of the CF 1A (mannitol 3%) and 3A (mannitol 3% & lactose 1%) were retained for additional appearance assessment over time to gather hold time information for discoloration without antioxidant. Vials of each formulation were stored under accelerated ambient light/temperature, and low light/temperature conditions and tested for appearance up to 96 hours (and 14 days).

No change in color was observed over 96 hours for all formulations without antioxidant (sodium bisulfite that used in previous formulation), have the appearance of a clear colorless liquid, free of visible particles,

Additionally, after storage for a further 14 days, the vials were inspected once again. The solution contained in vials that had been stored under low temperature/light conditions retained a colorless appearance, whilst those that had been stored under ambient light and temperature conditions exhibited a red/orange coloration. The coloration of formulation 3A, which contained lactose, appeared to be more advanced than that observed for formulation 1A, in which lactose was absent.

The application of an extended secondary drying step at +45° C. resulted in a lower residual moisture content for both CF1A2 and CF3A2, compared with their counterparts subjected to a standard secondary drying regimen. It is also notable that the CF3A1 and CF3A2 formulations, which contain 1% lactose as an excipient in addition to 3% mannitol, both exhibited slightly higher moisture content as compared with their CF1A counterparts containing mannitol only. This further supports the hypothesis that the higher residual moisture content observed in product from optimization cycle1 was the result of the formation of mannitol hemihydrate, rather than being associated with micro-collapse and, thus, indicates that the inclusion of lactose in the formulation is not necessary for the reduction of the residual moisture.

The lowest moisture content was exhibited by CF1A2 at 0.64% (mean), though that of CF1A1 was only slightly higher than this at 0.91% (mean) as shown in Table 10. This suggests that the use of a higher annealing temperature is sufficient to avoid the formation of mannitol hemihydrate and that the inclusion of a +45° C. secondary drying step is unlikely to yield a product with a substantially improved residual moisture that would justify exposure of the product to the higher secondary drying temperature.

### Final Assessment Cycle

The mannitol based formulation proved to be the good candidate when using the optimized final lyophilization cycle, based on the low water content (less than 1%) and based on the mDSC analysis, which showed a higher glass transition temperature, which might allow for storage even in ambient condition for long term.

Analysis of mannitol-based samples indicated that the use of faster cooling and a higher annealing temperature was sufficient to prevent MHH and that use of the extended secondary drying step would be unnecessary for this formulation. Therefore, the mannitol based formulation was selected as the candidate and brought forward to the assessment/confirmation cycle. The concentration of buffering components was kept the same as that which was used for the optimization cycle 2, and the sodium bisulphite was omitted from all presentations.

Both the 6 mg dose and 1 mg dose presentation of CF1 were investigated in the assessment cycle, designated as CF1A (6 mg/vial) and CF1B (1 mg/vial), respectively. The concentration of buffering components was kept the same as those used for optimization cycle 2 and the sodium bisulphite was omitted from all presentations. A summary of the compositions of each of the candidate formulations is provided in Table 11.

The final assessment cycle was conducted using the established cycle conditions to yield a final product for subsequent use in a long-term stability study for confirmation of the product suitability. Pre- and post-lyophilization analytical analysis was performed to assess the product appearance, physicochemical characteristics, and thermostability characteristics.

The psilocin benzoate formulation that was determined to be most suitable for freeze-drying and a freeze-drying recipe established for both the low dose and high dose presentation of the product, is provided in Table 12.

A summary of the Psilocin Benzoate formulation composition of candidate formulations is provided in Table 11.

TABLE 11
Compositions of Candidate Final Formulations Investigated in Assessment Cycle

| | Psilocin Benzoate* | | | | |
|---|---|---|---|---|---|
| | Quantity | | Quantity per | Excipient conc. | |
| CF | (mg/mL) | | vial (mg/vial) | (%) | Buffer |
| CF 01A | 3 | mg/mL | 6 mg | Mannitol (3%) | 25 mM Citrate buffer: 2.6 mg/mL |
| CF 01B | 0.5 | mg/mL | 1 mg | | Citric Acid, 3.7 mg/mL Trisodium |
| | | | | | Citrate; pH 4.5- |

The labelled strength of DP bulk vials (1 mg-6.0 mg) represents the weight of the psilocin benzoate DS per vial dose calculated as free base. The DP contains psilocin benzoate equivalent to 1-6 mg per 2 ml of reconstituted solution (0.5-3.0 mg per mL-1-6 mg/2 ml per vial).
Theoretical quantity, which includes “overfill” 10% excess material. The exact quantity is calculated per batch to compensate for potency value and salt factor on the certificate of analysis for the input batch of Psilocin Benzoate DS.

$$The salt factor = 1.6 base to salt .$$

Water for Injection used as the process solvent, removed during freeze-drying cycle. Quantity added up to total fill volume.

A 10% excess volume overfill is included in the vial to assure withdrawal of the complete dose.

Sterile saline for injection serves as a diluent for the preparation of the final reconstituted/diluted solution for infusion.

Based on the lyophilization development studies the optimal formulation and lyophilization cycle was selected (see above). The summary of the parameters of the final freeze-drying recipe are provided in Table 12.

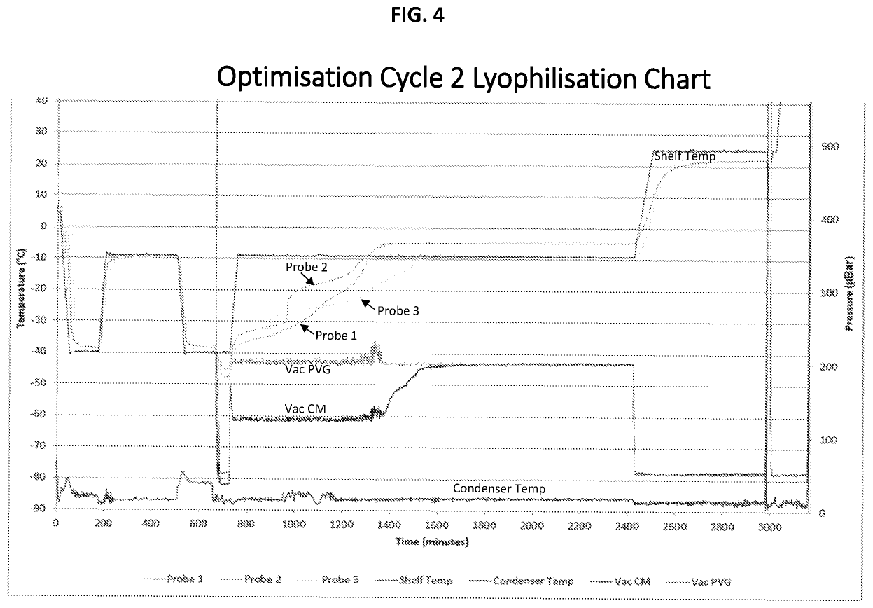

The representative optimization cycle 2 freeze-drying chart is provided in FIG. 4.

TABLE 12
Summary of the parameters of the recommended freeze-drying recipe

| | | | | |
|---|---|---|---|---|
| Step | Shelf temp | Time (mins) | Pressure | Description |

THERMAL TREATMENT STAGE

| | | | | | |
|---|---|---|---|---|---|
| 1 | 5° | C. | n/a | Atmospheric | Hold for product loading |
| 2 | −40° | C. | 45 | Atmospheric | Ramp |
| 3 | −40° | C. | 120 | Atmospheric | Hold |
| 4 | −9° | C. | 31 | Atmospheric | Ramp |
| 5 | −9° | C. | 300 | Atmospheric | Hold |
| 6 | −40° | C. | 31 | Atmospheric | Ramp |
| 7 | −40° | C. | 120 | Atmospheric | Hold |

PRIMARY DRYING CHECKPOINT PARAMETERS

| | | |
|---|---|---|
| Freeze Temperature: −40.0° C. | Vacuum setpoint: 500 μBar | Condenser setpoint: −60.0° C. |

PRIMARY DRYING STAGE

| | | | | | | |
|---|---|---|---|---|---|---|
| 8 | −40° | C. | 60 | 50 | μBar | Hold |
| 9 | −9° | C. | 31 | 200 | μBar | Ramp |
| 10 | −9° | C. | 2566 | 200 | μBar | Hold |

SECONDARY DRYING STAGE

| | | | | | | |
|---|---|---|---|---|---|---|
| 11 | +25° | C. | 80 | 50 | μBar | Ramp |
| 12 | +25° | C. | 480 | 50 | μBar | Hold |

ACTIONS AT END OF CYCLE

| | | | | | |
|---|---|---|---|---|---|
| 13 | +25° | C. | N/A | Half Atmospheric | Backfill with N₂ and Stopper all vials |
| 14 | +25° | C. | N/A | Half Atmospheric | Aerate freeze-dryer and unload vials |

Total cycle duration: ~63 hours

Tables 13 & 14 provide the range of results for the parameters evaluated during the development for the chosen final lyophilization cycle in the following conditions
a) Pre-lyophilized solution stability
b) After lyophilization, initial time point
c) After 4 weeks storage at 25° C./60% RH and 40° C./75% RH

The results of post-lyophilization thermal analysis and moisture analysis are provided in Table 13.

TABLE 13
Summary of Post-lyophilization physicochemical properties

| Physical/Chemical | |
|---|---|
| Property | Reported Findings |
| Appearance Properties | Lyophilized Psilocin benzoate DP cakes are white to off-white in color. The |
| | cakes were found to be overall compact with no shrinkage, no crumbling. The |
| | lyophilizates were cylindrical in shape and had a good contact with the |
| | container wall and bottom. The surface was smooth and shiny. Upon storage |
| | in accelerated conditions for 4 weeks, no change in cake appearance was |
| | detected. |
| | Pre-lyophilised solution of the candidate formulation without antioxidant were |
| | clear and colorless liquid, free of visible particles. No change in solution |
| | appearance was detected upon storage in accelerated conditions (under |
| | ambient light/ temperature) for 96 hours. |
| | Reconstituted solution in diluent were clear and colorless liquid, free of visible |
| | particles. No change in solution appearance was detected upon storage in |
| | accelerated conditions (under ambient light/temperature) for 24 hours, and |
| | in refrigerated (at 2-8° C.) for 24 days. |
| | The results provide an indication of liquid state stability and product hold time |
| | information for consideration as formulations during scale up may spend a |
| | significant amount of time during filling before being loaded into the freeze |
| | dryer and after reconstitution for product in-use stability. |
| Water Content | 0.7% |
| MDSC Analysis | Glass Transition Temp (Midpoint) |
| | Tg at 68.73° C. to 72.57° C. |
| Reconstitution time | <60 seconds |
| pH | 4.5 |

A summary, stability data of the psilocin benzoate formulation composition of high concentration 6 mg/vial candidate formulations is provided in Table 14.

TABLE 14
Summary of the analytical results

| | Acceptance | Initial time | | 4 week time point | | | | | |
|---|---|---|---|---|---|---|---|---|---|
| Test | Criteria | point | | 2-8° C. | | 25° C./60% RH | | 40° C./75% RH | |
| Appearance | White | conforms | | conforms | | conforms | | conforms | |
| | lyophilized | | | | | | | | |
| | powder in a | | | | | | | | |
| | 6 mL glass | | | | | | | | |
| | vial. | | | | | | | | |
| Appearance | A clear, | A clear, colorless | | A clear, | | A clear, colorless | | A clear, colorless solution | |
| of | colorless | solution | | colorless | | solution | | | |
| reconstituted | liquid, free | | | solution | | | | | |
| solution and | of visible | | | | | | | | |
| visible | particles | | | | | | | | |
| particles | | | | | | | | | |
| Completeness | Dissolves | conforms | | conforms | | conforms | | conforms | |
| of | completely | | | | | | | | |
| reconstituted | in 2 mL | | | | | | | | |
| solution and | diluent | | | | | | | | |
| dissolution | within 60 | | | | | | | | |
| time | sec | | | | | | | | |
| pH of | 3.5-5.0 | 4.5 | | 4.6 | | 4.6 | | 4.7 | |
| reconstituted | | | | | | | | | |
| solution | | | | | | | | | |
| Water | NMT 5.0%. | 0.7% | | 0.79% | | 0.95% | | 1.05% | |
| content KF | | | | | | | | | |
| (Oven) | | | | | | | | | |
| Assay (of DS | 90.0%- | 99.9% | | 101.6% | | 101.2% | | 101.5% | |
| labeled | 110.0% | | | | | | | | |
| amount) | | | | | | | | | |
| | | RRT | % Area | RRT | % Area | RRT | % Area | RRT | % Area |
| Related | NMT 1.0% | 1.21/1.23 | 0.20 | 1.21 | 0.18 | 1.21 | 0.17 | 1.21 | 0.17 |
| Substances | Total: NMT | 1.85 | 0.10 | 1.23 | 0.18 | 1.23 | 0.18 | 1.23 | 0.17 |
| Unspecified | 5.0% | Impurities | 0.30 | 1.85 | 0.10 | 1.85 | 0.10 | 1.85 | 0.11 |
| impurities, each | | Total | | Impurities | 0.46 | Impurities | 0.45 | Impurities | 0.45 |
| Total | | % Area | | Total | | Total | | Total | |
| impurities | | | | % Area | | % Area | | % Area | |

At the 4 week time point, the appearance, assay concentration, related substances and water content, for all storage conditions were as expected and well within the acceptance criteria.

The appearance/pH, reconstitution time results were presented and confirmed that all criteria have been met for all storage conditions.

Assay and related substances results showed that the assay/impurities are not affected at 2-8° C. and 25° C./60% RH, 40° C./75% RH, up to 4 weeks storage and are comparable to initial time point.

Assay did not decrease at all conditions. at the two-week timepoint, the purity exceeds 98% by relative area for all replicates.

There was no trending for any impurity peaks against conditions. The consistently observed peaks at RRT's 1.21; 1.23; 1.85 did not increase from time zero and is also present in the API refence standard material at similar levels.

### Example 2. Non Clinical Studies with Psilocin Benzoate Formulation

An assessment of local tolerance of the psilocin benzoate formulation following IV infusion administration has been conducted in Wistar rats and Beagle dogs. In an initial study, 3 male Wistar rats were administered a single dose of the psilocin benzoate formulation at a dose of 1 mg/kg, 1 mL/kg, or 1 mg/mL. No mortality, injection site reactions, injection site macropathology or histopathology findings were observed. The no-adverse-effect-level (NOAEL) for local tolerance in male Wistar rats was considered to be 1 mg/kg (1 mg/mL, 1 mL/kg, 10-minute IV infusion) in the revised formulation, and effects were consistent with those observed for the original (Phase 1/2a) formulation. A comparison of the Original and Revised formulations is shown in Table 3.

In an initial maximum tolerant dose (MTD) study, 1 male and 1 female beagle dog were administered a single dose of the psilocin benzoate revised formulation at a dose of 1 mg/kg, 5 mL/kg, 0.2 mg/mL as part of a dose escalation design of different vehicles and infusion durations. No mortality or injection site reactions were observed during dosing with either original or revised formulations. There were no psilocin benzoate related macroscopic or microscopic findings. Injection site findings were considered most likely to be procedural rather than directly related to the test item. The NOAEL for local tolerance in male and female beagle dogs was considered to be 1 mg/kg (5 mL/kg, 0.2 mg/mL) in the Phase 2b formulation.

### OTHER EMBODIMENTS

All publications, patents, and patent applications mentioned in this specification are herein incorporated by reference to the same extent as if each independent publication or patent application was specifically and individually indicated to be incorporated by reference. The content of U.S. provisional Ser. No. 63/148,052, filed Feb. 10, 2021, and 63/276,096, filed Nov. 5, 2021, is incorporated herein by reference in its entirety.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims. Other embodiments are within the claims.

What is claimed is:

1. A lyophilized solid comprising:
(i) amorphous psilocin benzoate;
(ii) citrate buffer as a buffering agent;
(iii) mannitol as a bulking agent; and
(iv) less than 2% (w/w) water.

2. The lyophilized solid of claim 1, wherein the psilocin benzoate has a concentration of between 0.01% (w/w) and 10% (w/w).

3. The lyophilized solid of claim 1 or 2, wherein the solid comprises less than 80% (w/w) of the buffering agent.

4. The lyophilized solid of claim 1 or claim 2, wherein the solid comprises between 10% (w/w) and 95% (w/w) or between 10% (w/w) and 30% (w/w) buffering agent.

5. The lyophilized solid of claim 4, wherein the bulking agent has a concentration of between 65% (w/w) and 95% (w/w) or between 70% (w/w) and 90% (w/w).

6. The lyophilized solid of any one of claims 1-5, wherein the solid comprises less than 1% (w/w) water.

7. The lyophilized solid of any one of claims 1-6, wherein the solid has a pH of between 3.5 and 5 when dissolved in between 10 mL and 50 mL of unbuffered water or saline for injection.

8. The lyophilized solid of any one of claims 1-7, wherein the solid further comprises less than 1% (w/w) of an antioxidant.

9. The lyophilized solid of claim 8, wherein the antioxidant is sodium bisulfite.

10. The lyophilized solid of any one of claims 1-7, wherein the solid is free of any antioxidant.

11. The lyophilized solid of any one of claims 1-10, wherein the solid comprises less than 20% (w/w) by weight sodium chloride.

12. The lyophilized solid of any one of claims 1-10, wherein the solid is free of sodium chloride.

13. The lyophilized solid of any one of claims 1-12, wherein the lyophilized solid does not comprise mannitol hemihydrate.

14. The lyophilized solid of any one of claims 1-12, wherein the lyophilized solid is substantially free of mannitol hemihydrate.

15. The lyophilized solid of any one of claims 1-12, wherein the lyophilized solid is substantially free of mannitol hemihydrate and wherein the lyophilized solid is characterized by an X-ray powder diffractogram (XRPD) which does not comprise a peak at the 20 values of 17.9±0.1°, 17.9±0.2° or 17.9±0.3° as measured using an X-ray wavelength of 1.5406 A.

16. A method of preparing a lyophilized solid comprising:
(i) amorphous psilocin benzoate;
(ii) a buffering agent; and
(iii) less than 3% (w/w) water,
wherein the solid of any one of claims 1-16 is dissolved in between 5 mL to 50 mL of an aqueous solution and undergoes a thermal treatment step, followed by a drying step.

17. The method of claim 16, wherein the thermal treatment step is performed for between 30 hours and 70 hours at a temperature of between −50° C. and 10° C.

18. The method of claim 17, wherein the thermal treatment step comprises:
(i) a first step, wherein the first step is performed at about 5° C. for about 10 minutes;
(ii) a second step, wherein the second step is performed at about −40° C. for about 45 minutes;
(iii) a third step, wherein the third step is performed at about −40° C. for about 120 minutes;
(iv) a fourth step, wherein the fourth step is performed at about −9° C. for about 31 minutes;
(v) a fifth step, wherein the fifth step is performed at about −9° C. for about 300 minutes;
(vi) a sixth step, wherein the sixth step is performed at about −40° C. for about 31 minutes; and
(vii) a seventh step, wherein the seventh step is performed at about −40° C. for about 120 minutes.

19. The method of any one of claims 16-18, wherein the drying step comprises a primary drying step and a secondary drying step.

20. The method of claim 19, wherein the primary drying step is performed for between 5 hours and 10 hours at a temperature of between −50° C. and 0° C., and a pressure of between 20 μBar and 300 μBar.

21. The method of claim 20, wherein the primary drying step comprises a first step, a second step, and a third step, wherein the first step is performed at about −40° C. for about 60 minutes with a pressure of about 50 μBar, wherein the second step is performed at about −9° C. for about 31 minutes with a pressure of about 200 μBar, and wherein the third step is performed at −9° C. for about 2566 minutes with a pressure of about 200 μBar.

22. The method of claim 20 or 21, wherein the secondary dry step comprises a first step and second step, wherein the first step is performed at 25° C. for about 78 minutes at a pressure of about 50 μBar and wherein the second step is performed at 25° C. for about 480 minutes at a pressure of about 50 μBar.

23. A lyophilized solid of any one of claims 1-15 dissolved in from 5 mL to 50 mL of an aqueous solution for use in a method of treating a disease or condition in a subject in need thereof, the method comprising intravenously administering to the subject the lyophilized solid dissolved in from 5 mL to 50 mL of an aqueous solution in an amount sufficient to treat the disease or condition.

24. The lyophilized solid for use of claim 23, for use in a method of treating major depression, treatment resistant depression, melancholic depression, atypical depression, dysthymia, end of life anxiety, generalized anxiety disorder, panic disorder, social anxiety, post-traumatic stress disorder, acute stress disorder, obsessive compulsive disorder, social phobia, substance abuse, alcoholism, tobacco abuse, drug abuse, anorexia nervosa, bulimia nervosa, binge eating disorder, primary impulse-control disorders or obsessive-compulsive disorder.

25. The lyophilized solid for use of claim 24, for use in a method of treating major depression.

26. The lyophilized solid for use of claim 24, for use in a method of treating treatment resistant depression.

27. The lyophilized solid for use of claim 24, for use in a method of treating anxiety.

28. The lyophilized solid for use of claim 24, for use in a method of treating addiction.
